(19) United States
(12) Patent Application Publication
(10) Pub. No.: US 2025/0043293 A1
(43) Pub. Date: Feb. 6, 2025

(54) BACILLUS EXPRESSION SYSTEM

(71) Applicant: WILLIAM MARSH RICE UNIVERSITY, Houston, TX (US)

(72) Inventors: Jeffrey J. TABOR, Houston, TX (US); Sebastian M. CASTILLO-HAIR, Houston, TX (US)

(21) Appl. No.: 17/311,675

(22) PCT Filed: Dec. 6, 2019
(86) PCT No.: PCT/US19/65080

§ 371 Date: Jun. 7, 2021

Related U.S. Application Data
(60) Provisional application No. 62/776,132, filed on Dec. 6, 2018.

(51) Int. Cl. C12N 15/75; C12N 1/20; C12N 15/63; C12R 1/125

(52) U.S. Cl. CPC C12N 15/75; C12N 1/20; C12N 15/635; C12R 2001/125

(57) ABSTRACT

An inducible promoter expression system based on the T7 RNA Polymerase (T7 RNAP), lactose repressor (Lad), and a chimeric T7lac promoter (PT7lac), which can be integrated as a single copy into the B. subtilis genome. In the absence of IPTG. Lad strongly represses T7RNAP and PT7lac, and expression of an exemplary ORF—here superfolder green fluorescent protein (sfGFP) reporter protein—is undetectable by flow cytometry. Addition of IPTG de-represses PT7lac, and simultaneously induces expression of T7RNAP, resulting in very high sfGFP levels.

### PRIOR RELATED APPLICATIONS

This application claims priority to U.S. Ser. No. 62/776,132, filed Dec. 6, 2018, and incorporated by reference in its entirety for all purposes.

### FEDERALLY SPONSORED RESEARCH STATEMENT

This invention was made with government support under Grant Nos: R21AI115014 and MCB1616755, awarded by the NIH and NSF, respectively. The government has certain rights in the invention.

### FIELD OF THE DISCLOSURE

This invention provides materials and methods allowing expression of RNA and proteins in Bacillus, especially B. subtilis and similar species, with a new tightly regulated, integrative and inducible expression system.

### BACKGROUND OF THE DISCLOSURE

Bacillus subtilis is a model organism for studies of Gram-positive bacterial biology and systems biology of cellular differentiation, stress responses, and multicellular organization. Additionally, B. subtilis is among the most widely used hosts for protein production in the biotechnology industry due to its ability to secrete proteins into the cell medium, its non-pathogenic GRAS (generally recognized as safe) designation, and its high genetic tractability. For example, B. subtilis is used for large-scale production of lipases, proteases, and amylases, among other industrially-relevant proteins.

However, B. subtilis work has been limited by a lack of high-quality synthetic biological parts. For example, biotechnology applications require inducible promoters capable of switching between a low production state for early-stage culturing and a high production state that maximizes protein yield during fermentation. Such parts are particularly important when the recombinant protein or metabolic pathway of interest are toxic to the host cells when overproduced.

Typical inducible promoters in B. subtilis have dynamic ranges (ratio of output protein expression in the presence versus absence of inducer) of at most a few hundred. While a B. subtilis bacitracin-inducible promoter with a 1,000-fold range has been reported (13), it requires antibiotic selection to maintain a multicopy plasmid, and its activity is transient and shuts down less than two hours after induction, likely due to an endogenous bacitracin stress response. In contrast, for the Gram-negative bacterium Escherichia coli, inducible promoters have been engineered with dynamic ranges greater than 1,000 or even 10,000.

Thus, improved B. subtilis inducible promoter systems are greatly needed in the art. The ideal system will be very tightly regulated, result in high levels of protein expression when induced, have 10,000 or even greater fold dynamic range, and ideally be integratable into the genome.

### SUMMARY OF THE DISCLOSURE

Protein expression systems that are activated in response to chemicals such as IPTG, xylose or bacitracin currently exist for B. subtilis. However, their fold-activation values are fairly low, and their active output levels are limited by the B. subtilis endogenous transcription machinery. On the other hand, a few systems based on the exogenous viral T7 RNA Polymerase (T7 RNAP) have been previously built. While these achieve high expression levels, protein production in the inactive state or “leakiness” is fairly high and therefore fold-activation is still low.

Here, we have engineered an extraordinarily stringent and strongly inducible protein expression system for Bacillus. This system combines the viral T7 RNAP, which produces high amounts of recombinant protein in the active state, with the LacI bacterial transcription factor that inhibits production of both the protein and the viral polymerase, resulting in nearly undetectable output in the inactive state. Our system achieves greater than 10,000-fold or even 20,000-fold activation in response to a chemical inducer. We expect this system to improve yield of existing protein production, especially toxic proteins, or metabolic engineering applications using B. subtilis and possibly other Bacillus species, and enable synthesis of new proteins and chemicals that were previously unfeasible.

Our LacI-T7 inducible system comprises the following elements:
Transcriptional repressor LacI, expressed constitutively.
The T7 phage RNA polymerase (T7 RNAP), expressed from the B. subtilis promoter P_{hy-spank}, which is normally repressed by LacI.
The hybrid P_{T7lac} promoter, normally repressed by LacI as well, under whose control the recombinant protein of interest or ORF is placed.

For an integrative sequence, these elements should be flanked by first and second portions of a non-essential portion of the Bacillus genome, thereby allowing integration into that genome. The order of parts is typically not critical, but whatever the order, the cassette is flanked by the integrating sequences such that everything there between is integrated into the genome. In addition, sequences to optimize expression of the target ORF can also be included, such as ribosome binding sites, enhancers, terminators, and the like.

The cassette can be assembled and used as is, or can be formulated as an expression vector—capable of self-reproduction and ORF expression. For an expression vector, additional vector sequences are added, allowing replication in Bacillus and/or E. coli (e.g., ori), marker genes for selection (e.g., antibiotic resistance), unique restriction endonuclease (RE) sites or the multi-cloning site, shuttle sequences allowing the vector to shuttle e.g., between E. coli and Bacillus (both ori sequences), or between yeast and Bacillus (Bacillus ori and autonomously replicating sequence (ARS), a yeast centromere (CEN), and a yeast selectable marker), viral packaging sequences, protein degradation tags, and the like. It may also be possible to formulate the cassette described herein as minivectors, such as those described by Twister® (Houston TX).

LacI and the P_{hy-spank} promoter were obtained from pDR111 (ATCC® 53123), a commercially available plasmid for genomic integration in B. subtilis. The P_{T7lac} promoter sequence is identical to that included in some of the commercially-available E. coli pET plasmids, and was synthesized via oligo annealing and extension. The sequence of the T7 RNAP is identical to that found in the E. coli BL21 (DE3) strain, and was obtained from an E. coli plasmid.

DNA coding for these elements was assembled, together with a spectinomycin resistance cassette. Flanking sequences for genome integration were added that were homologous to the B. subtilis amyE genomic locus, but any non-essential loci could be used. The assembled DNA cassette was then integrated into the Bacillus genome via standard Bacillus transformation methods.

In the inactive state, LacI repression of both T7 RNAP production and P_{T7lac} ensures very low production of recombinant protein. Upon addition of chemical inducer IPTG to the cell media, LacI-repression is relieved and the T7 RNAP is produced. The T7 RNAP can now transcribe the recombinant gene from the now unrepressed P_{T7lac} promoter.

To assess the performance of the LacI-T7 system, we placed superfolder GFP (sfGFP) under P_{T7lac} and measured cell fluorescence in the presence or absence of IPTG. This resulted in high levels of fluorescence in the presence of IPTG, but undetectable levels in its absence. Based on estimates of the limit of detection of our flow cytometer instrument, we placed a lower bound on the fold-activation of this system at 20,000. We have also tested the system with other report

The invention includes any one or more of the following in any combination(s) thereof:

An inducible and integrative genetic system for Bacillus subtilis, said system comprising
the following components:
a) a Lacl repressor constitutively expressed under a first constitutive promoter from B.
subtilis;
b) a T7 phage RNA polymerase (T7 RNAP) expressed from a second B. subtilis promoter
that is normally repressed by Lacl;
c) a hybrid output promoter activated by T7RNAP and repressed by Lacl;
d) part a) being 5′ flanked by a first portion of a chromosomal gene from B. subtilis, and
part c) being 3′ flanked by a second portion of said chromosomal gene from B. subtilis, so as
to allow integration into a genome of B. subtilis.
A system as herein described, having at least one open-reading frame under the control of
the output promoter.
A system as herein described, having at least 10,000 fold or 20,000 activation (expression of
said ORF before and after induction) when induced.
The system can also include other sequences for convenience of use, such as E. coli ori,
antibiotic resistance cassette, multiple cloning sites, and the like, and thus be able to shuttle
from species to species and/or function as a vector for amplification and cloning purposes.
Herein, however, we assembled the components in vitro and then used the assembled
material for direct integration.
A system as herein described, wherein the Lacl was obtained from or is equivalent to the
sequences in pDR111.
A system as herein described, wherein said first promoter is a P_{penP} promoter.
A system as herein described, wherein said second promoter is a P_{hy-spank} promoter.
A system as herein described, wherein the P_{hy-spank} promoter was obtained from or is
equivalent to the sequences in pDR111.
A system as herein described, wherein the P_{T7lac} promoter sequence was obtained from or
is equivalent to E. coli pET plasmids.
A system as herein described, wherein the T7 RNAP was obtained from or is equivalent to
the E. coli BL21(DE3).
A system as herein described, further comprising an antibiotic resistance cassette, e.g., a
spectinomycin resistance cassette or others.
A system as herein described, wherein said chromosomal gene from B. subtilis is an
amyE gene.
An expression system for Bacillus subtilis, said system having a structure of FIG. 1A.
An expression system for Bacillus subtilis, said system having a sequence of SEQ ID NO
7, or at least 95% identity to same
An inducible and integrative expression system for Bacillus subtilis, said system
comprising the following components:
a) a Lacl repressor constitutively expressed under a B. subtilis P_{penP} promoter;
b) a lacl-repressible T7 phage RNA polymerase (T7 RNAP) expressed from a B. subtilis
P_{hy-spank} promoter;
c) a hybrid P_{T7lac} promoter regulated by said T7RNAP;
d) a multiple cloning site for insertion of an ORF operably linked to said P_{T7lac} promoter;
e) part a) being 5′ flanked by a first portion of a chromosomal gene from B. subtilis, and
part d) being 3′ flanked by a second portion of said chromosomal gene from B. subtilis, so as
to allow integration into a genome of B. subtilis.
A recombinant Bacillus, wherein said Bacillus is transformed with a system as herein
described, said system having an ORF for a target protein inserted after P_{T7lac}, said Bacillus
able to express said target protein when induced with IPTG.
A recombinant Bacillus, wherein said Bacillus is transformed with the system herein
described, said system having an ORF for a target protein inserted after P_{T7lac}, said ORF being
integrated into a genome of said Bacillus, and said Bacillus able to express said target protein
when induced with IPTG.
A method of transforming Bacillus subtilis, comprising introducing the system as herein
described to a population of Bacillus subtilis under conditions that allow transformation and
integration of said system into the genome of said Bacillus subtilis, and selecting for
transformed and integrated Bacillus subtilis.
A method of producing a target protein, comprising introducing an ORF encoding a target
protein after P_{T7lac} of the system as herein described, introducing said system and ORF to a
population of Bacillus subtilis under conditions that allow integration of said ORF into a
genome of said Bacillus subtilis, and selecting for integrated Bacillus subtilis, growing said
integrated Bacillus subtilis in a growth medium until cells reach near saturation, adding IPTG
to said growth medium in an amount sufficient to induce the lac operon, continue culturing said
integrated Bacillus subtilis until said ORF is expressed, and isolating said target protein from
said growth medium or said transformed Bacillus subtilis or both.
A Bacillus promoter system, comprising an DNA molecule comprising:
a) a Lacl repressor constitutively expressed under a first constitutive promoter;
b) a T7 phage RNA polymerase (T7 RNAP) expressed from a second promoter that is
normally repressed by Lacl; and
c) a third promoter sequence regulated by a T7RNAP that is normally repressed by Lacl.
Any promoter system herein, further comprising an open reading frame (“ORF”) for a gene
of interest inserted after said third promoter sequence, such that said ORF is regulated by
said third promoter sequence.
Any promoter system herein, having at least 5,000, 10,000, 15,000 or 20,000 fold activation
of expression of said ORF when induced by IPTG.
Any promoter system herein, comprising one or more of the following:
a) the Lacl is from or is equivalent to the sequences in pDR111 or having at least 95%
nucleotide identity to SEQ ID NO 11; or
b) the first constitutive promoter is a PpenP promoter or a sequence having at least 95%
nucleotide identity to SEQ ID NO 15; or
c) the second promoter is a Phy-spank promoter or a sequence having at least 95%
nucleotide identity to SEQ ID NO 13; or
d) the Phy-spank promoter is from or is equivalent to the sequences in pDR111; or
e) the hybrid PT7lac promoter sequence is from or is equivalent to E. coli pET plasmids;
or
f) the T7 RNAP is from or is equivalent to the E. coli BL21(DE3); or
g) the T7RNAP is a sequence having at least 95% nucleotide identity to SEQ ID NO 12
h) the third promoter is a hybrid PT7lac promoter sequence or a sequence having at least
95% nucleotide identity to SEQ ID NO 14; or
i) the first constitutive promoter for the Lacl repressor is a promoter from Bacillus; or
j) the T7RNAP is expressed from a Bacillus Lacl repressed promoter.
An expression system for Bacillus, said system being in a Bacillus and having:
a) at least 95% nucleotide identity to the sequence of SEQ ID NO 7, said 95% identity
excluding an open reading frame (ORF) added thereto; and
b) having at least 5,000, 10,000, 15,000 or 20,000 fold more expression of said ORF with
IPTG induction than without IPTG induction.
An inducible and integrative expression system for Bacillus subtilis, said system comprising
an expression cassette with the following structure:
a) a first portion of a chromosomal sequence from B. subtilis at a 5′ end of said
expression cassette;
b) a second portion of said chromosomal sequence at a 3′ end of said expression
cassette;
c) a Lacl repressor constitutively expressed under a B. subtilis PpenP promoter within
said expression cassette;
d) a lacl-repressible T7 phage RNA polymerase (T7 RNAP) expressed from a B. subtilis
Phy-spank promoter within said expression cassette;
e) a hybrid promoter activated by T7RNAP and repressed by Lacl that controls expression
of an ORF.
Any system herein, further comprising a cassette comprising an antibiotic resistance gene
or a spectinomycin resistance gene.
Any system herein, further comprising a cassette comprising a 5′ chromosomal sequence
from the Bacillus amyE gene and a 3′ chromosomal sequence from the Bacillus amyE gene
to allow homologous recombination into a genome of Bacillus.
Any promoter system herein, further comprising one or more B. subtilis-specific ribosome-
binding sites (RBSs) placed to stimulate translation from mRNAs expressed from the first,
second or third promoters and/or comprising one or more B. subtilis-specific ribosome-
binding sites (RBSs) placed to stimulate translation from mRNAs expressed from PT7lac,
Phy-spank, or PpenP promoters, or any combination thereof
Any system herein, said cassette further comprising a self-cleaving ribozyme placed after
PT7lac to improve output production.
Any system herein, further comprising a multiple cloning site after said third promoter (for
insertion of the ORF).
Any system herein, wherein said Bacillus is a Bacillus subtilis.
Any system herein, wherein the promoter system is an inducible promoter system or is an
inducible by IPTG.
Any system herein, wherein said ORF encodes an output mRNA, that may or may not
encode a protein.
Any system herein, comprising an ORF for a target protein and being capable of a 10,000
fold increase in expression of said ORF when induced with IPTG as compared to without
IPTG.
A vector comprising any system herein, said vector being self-reproduceable in a Bacillus,
Escherichia or yeast.
Any vector herein producing at least 10,000 fold more expression of said ORF in a Bacillus
transformed with said vector and induced with IPTG induction, as compared to without
IPTG.
A recombinant Bacillus, wherein said Bacillus is transformed with any system herein, said
system being integrated or not.
A method of transforming Bacillus subtilis, comprising introducing any system herein to a
population of Bacillus subtilis under conditions that allow transformation and integration of
said system into a chromosome of said Bacillus subtilis, and selecting for transformed and
integrated Bacillus subtilis.
A method of producing a target protein, comprising introducing any system herein to a
population of Bacillus subtilis under conditions that allow integration of said expression
cassette into a genome of said Bacillus subtilis, and selecting for integrated Bacillus subtilis,
growing said integrated Bacillus subtilis in a growth medium until cells reach near saturation,
adding IPTG to said growth medium in an amount sufficient to induce said lac operon,
continuing culturing said integrated Bacillus subtilis until said ORF is expressed, and
isolating said target protein from said growth medium or said transformed Bacillus subtilis or
both.

As used herein “recombinant” or “engineered” is relating to, derived from, or containing genetically engineered material. In other words, the genome was intentionally manipulated by humans in some way.

“Expression vectors” are used in accordance with the art-accepted definition of a plasmid, virus, cosmid, or other propagatable sequence designed for protein expression in cells. There are thousands of such vectors commercially available, and typically each has an origin of replication (ori); a multiple cloning site; a selectable marker; ribosome binding sites; a promoter and often enhancers; and the needed termination sequences. Most expression vectors are inducible, although constitutive expression vectors also exist and either can be used.

As used herein, “inducible” means that gene expression can be controlled by the hand-of-man, by adding e.g., a ligand to induce expression from an inducible promoter. Exemplary inducible promoters include the lac promoter, inducible by isopropylthio-β-D-galactopyranoside (IPTG), the yeast AOX1 promoter inducible with methanol, the strong LAC4 promoter inducible with lactate, and the like. Low level of constitutive protein synthesis may occur even in expression vectors with tightly controlled promoters. “Constitutive” means there is always expression from that promoter. P_{veg} is an example of same.

As used herein, “integrative” or “integratable” means the nucleic acid has the ability to integrate into a B. subtilis or equivalent genome.

As used herein, an “integrated sequence” means the sequence has been integrated into the host genome, as opposed to being maintained on an expression vector or as a separate integration cassette. It will still be expressible, either inducibly or constitutively. Herein, we are more interested in tightly regulated and highly activatable inducible expression vectors and/or cassettes that are integrative or are integrated.

As used herein, “operatively connected” or “operatively coupled” with respect to DNA sequences means that the oligonucleotide segments are connected in such a way as to allow the DNA (or RNA or protein derived therefrom) to be functional in a cell. Typically, this means e.g., the correct spacing, essential regulatory sequences, and reading frame (if applicable) are maintained.

As used herein, “homolog” means an enzyme with at least 40% identity to one of the listed sequences and also having the same general catalytic activity, although kinetic parameters of the reactions can of course vary. While higher identity (60%, 70%, 80%) and the like may be preferred, it is typical for bacterial sequences to diverge significantly (40-60% identity), yet still be identifiable as homologs, while mammalian species tend to diverge much less (80-90% identity). Unless specified otherwise, any reference to an enzyme herein also includes its homologs that catalyze the same reaction.

As used herein, references to cells or bacteria or strains and all such similar designations include progeny thereof. It is also understood that all progeny may not be precisely identical in DNA content, due to deliberate or inadvertent mutations that have been added to the parent. Mutant progeny that have the same function or biological activity as screened for in the originally transformed cell are included. Where distinct designations are intended, it will be clear from the context.

The use of the word “a” or “an” when used in conjunction with the term “comprising” in the claims or the specification means one or more than one, unless the context dictates otherwise.

The term “about” means the stated value plus or minus the margin of error of measurement or plus or minus 10% if no method of measurement is indicated.

The use of the term “or” in the claims is used to mean “and/or” unless explicitly indicated to refer to alternatives only or if the alternatives are mutually exclusive.

The terms “comprise”, “have”, “include” and “contain” (and their variants) are open-ended linking verbs and allow the addition of other elements when used in a claim.

The phrase “consisting of” is closed, and excludes all additional elements.

The phrase “consisting essentially of” excludes additional material elements, but allows the inclusions of non-material elements that do not substantially change the nature of the invention.

The following abbreviations are used herein:

| ABBREVIATION | TERM |
|---|---|
| CDS | Protein coding sequence. Equivalent to an ORF. |
| GFP | Green fluorescent protein |
| IM | Integration module |
| IPTG | Isopropyl β-D-1-thiogalactopyranoside. When IPTG is added, |
| | the lac repressor is inactivated, allowing the T7 polymerase to |
| | be used and therefore the protein of interest will be expressed. |
| Lacl | Lactose operon repressor, e.g. P03023 |
| Lacl-T7 | Lacl operatively coupled to the T7 phage RNA polymerase |
| ORF | Open-reading frame |
| P_{hy-spank} | Promoter hyper spank |
| P_{penP} | Promoter from the penicillinase gene (penP) of Bacillus |
| | licheniformis. |
| RBS | Ribosome binding site |
| sfGFP | Superfolder GFP. A constitutively fluorescent green |
| | fluorescent protein, published in 2005. |
| T7 RNAP | T7 phage RNA polymerase, e.g., P00573 |

### DESCRIPTION OF FIGURES

FIG. 1AF. LacI-T7 design and performance.

FIG. 1A Genetic device schematic of LacI-T7 with regulatory interactions shown.

FIG. 1B sfGFP fluorescence from LacI-T7 and Phy-spank in the absence or presence of IPTG. Fluorescence is shown in calibrated Molecules of Equivalent Fluorescein (MEFL) units. Bars represent the mean of three experimental replicates run on separate days. Dots show the values of the individual replicates.

FIG. 1C-D Representative flow cytometry histograms of autofluorescence control (B. subtilis lacking any sfgfp gene), and those with sfgfp under either LacI-T7 (1C) or P_{hy-spank} (1D), in the presence or absence of IPTG.

FIG. 1E sfGFP Fluorescence from LacI-T7 and P_{hy-spank} as a function of IPTG concentration.

FIG. 1F sfGFP fluorescence from LacI-T7 and P_{hy-spank} after addition of saturating IPTG. Dots and error bars show the mean and standard deviation, respectively, of three experiments run on separate days. Error bars are, in most cases, smaller than the size of the dots, and thus not visible. Black lines represent model fits.

FIG. 2: T7lac promoter (P_{T7lac}) and placing of the gene of interest. P_{T7lac} is highlighted with underline. Ribosome binding site is highlighted with double underline. The start codon (ATG) of the gene to be expressed is shown in bold. SEQ ID NO 1.

FIG. 3: Promoter hyper spank SEQ ID NO 2.

FIG. 4. T7 RNAP SEQ ID NO 3.

FIG. 5: Promoter penP SEQ ID NO 4.

FIG. 6. LacI-T7-sfgfp. SEQ ID NO 5.

FIG. 7: Compete expression system sequence SEQ ID NO 6.

FIG. 8 Complete Vector without sfGFP SEQ ID NO 7.

FIG. 9. Variant LacI-T7-sfgfp SEQ ID NO 8.

FIG. 10: Compete Vector with sfGFP Variant SEQ ID NO 9.

FIG. 11. Growth curves of LacI-T7 strains. LacI-T7-sfgfp (A) and LacI-T7-lacZ (B) strains were grown in the absence or presence of IPTG, and the OD600 of each culture was measured at the indicated timepoints. Dots indicate individual OD600 measurements. Continuous lines indicate best fits to an exponential growth model. Estimated doubling times and uncertainties (standard error) are indicated next to the corresponding growth curves.

FIG. 12 SDS-PAGE and immunoblot analysis of sfGFP expression from Phy-spank and LacI-T7. (A) Whole cell extracts of wild-type, Phy-spank-sfgfp and LacI-T7-sfgfp strains grown in the absence or presence of IPTG were analyzed by SDS-PAGE and stained with Coomassie brilliant-blue. The arrow on the right points to the expected sfGFP location. (B) sfGFP expression was further examined by immunoblot analysis using polyclonal anti-GFP antibodies. (C) As a loading control, expression of the constitutively expressed GA protein was examined by immunoblotting with anti-GA antibodies.

FIG. 13. Performance of LacI-T7 measured via β-galactosidase. (A) Device schematic of the LacI-T7 system expressing lacZ. (B) β-galactosidase activity in the absence or presence of IPTG. Bars show the mean of three experiments run on separate days. Dots show values of individual experimental replicates.

Table 1. Data.

Table 2. Various sequences SEQ ID NO 10-22.

### DETAILED DESCRIPTION

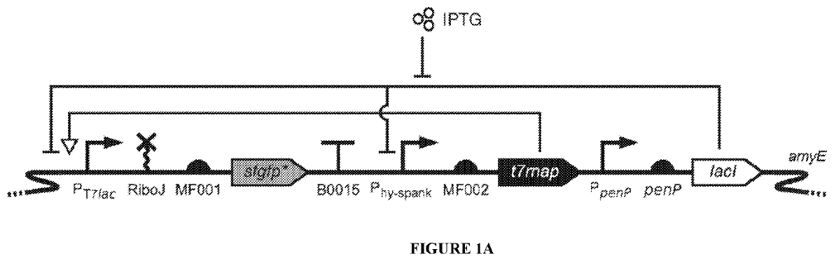

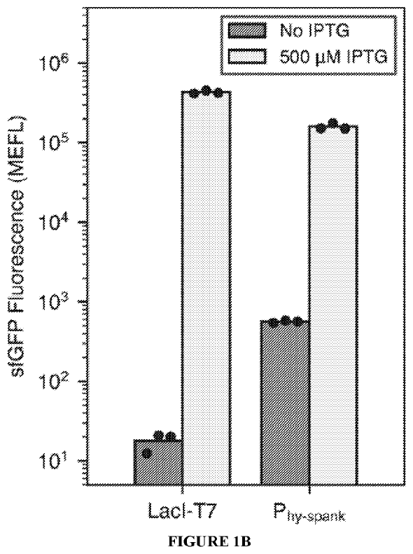

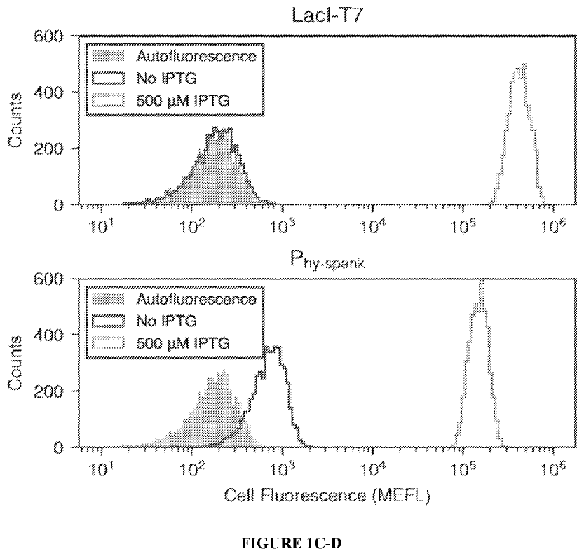

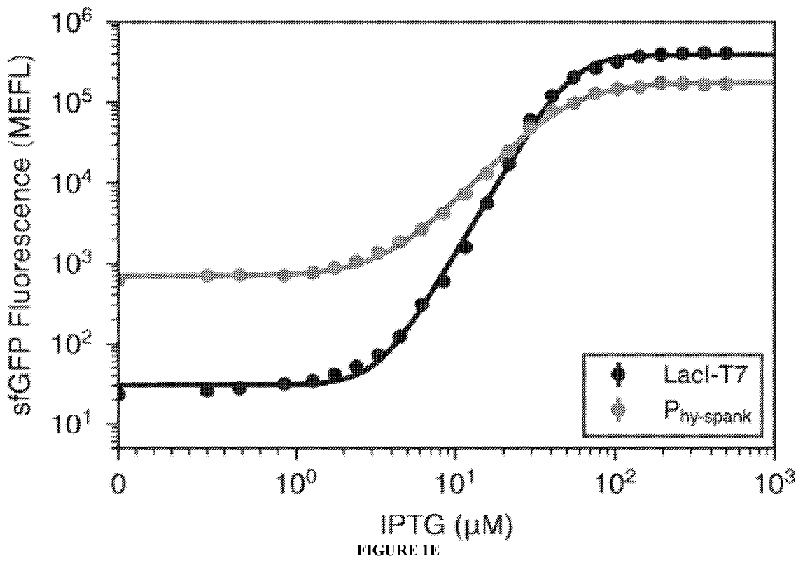

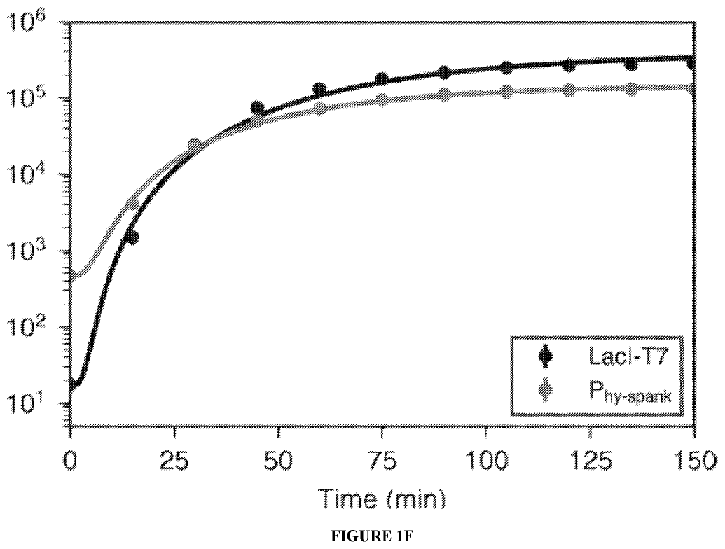

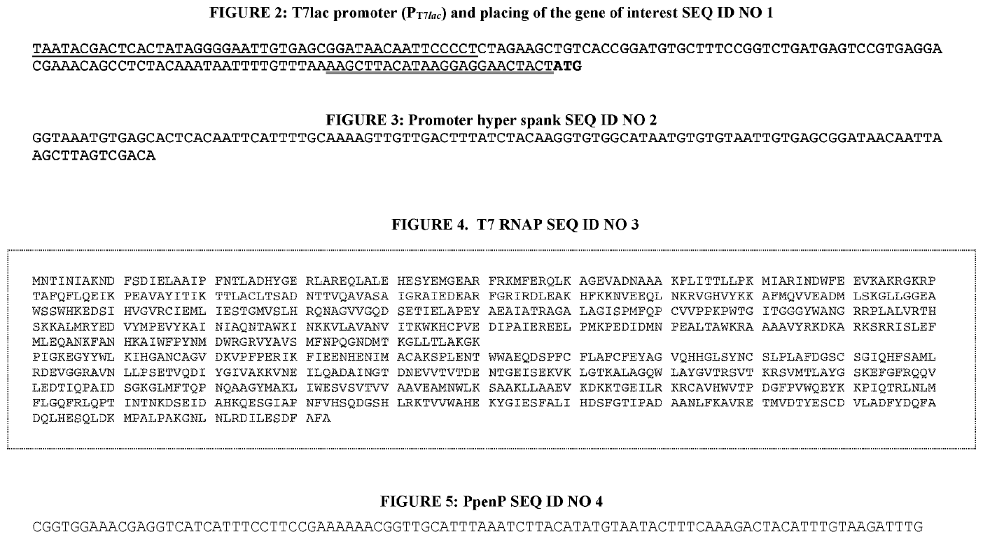

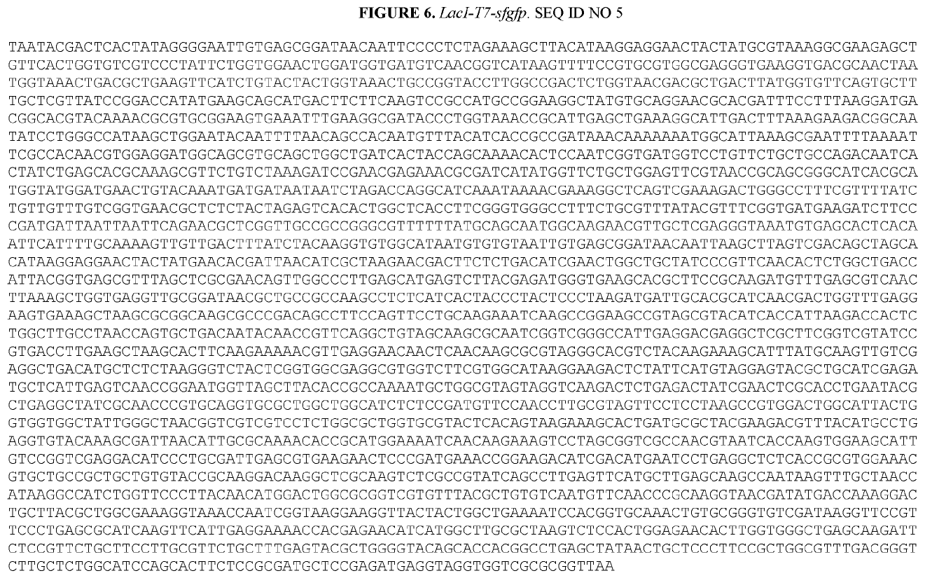

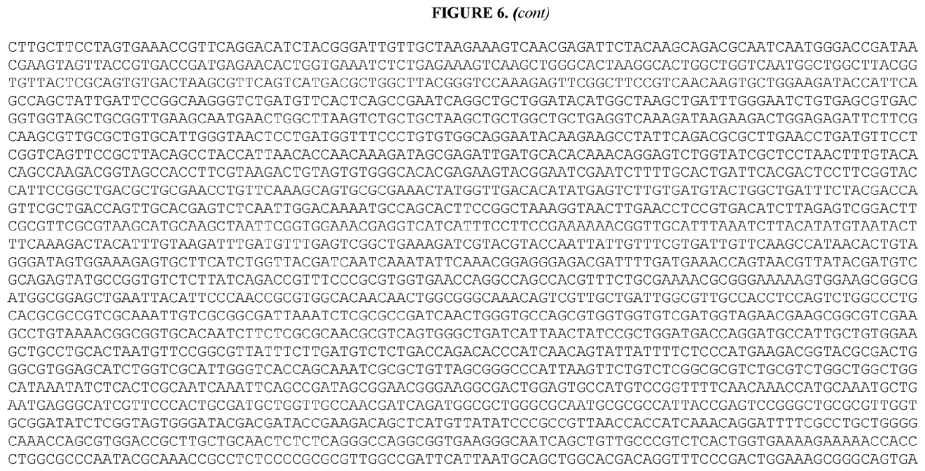

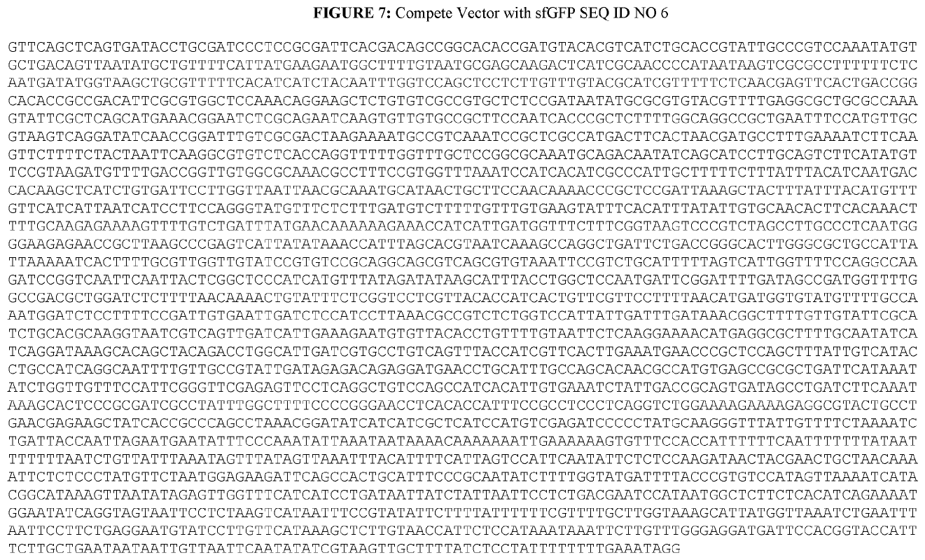

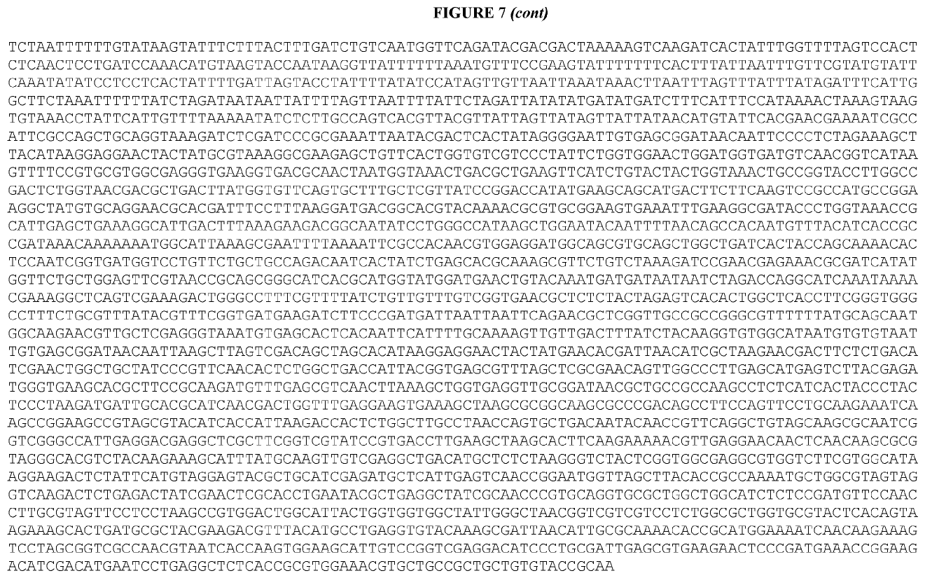

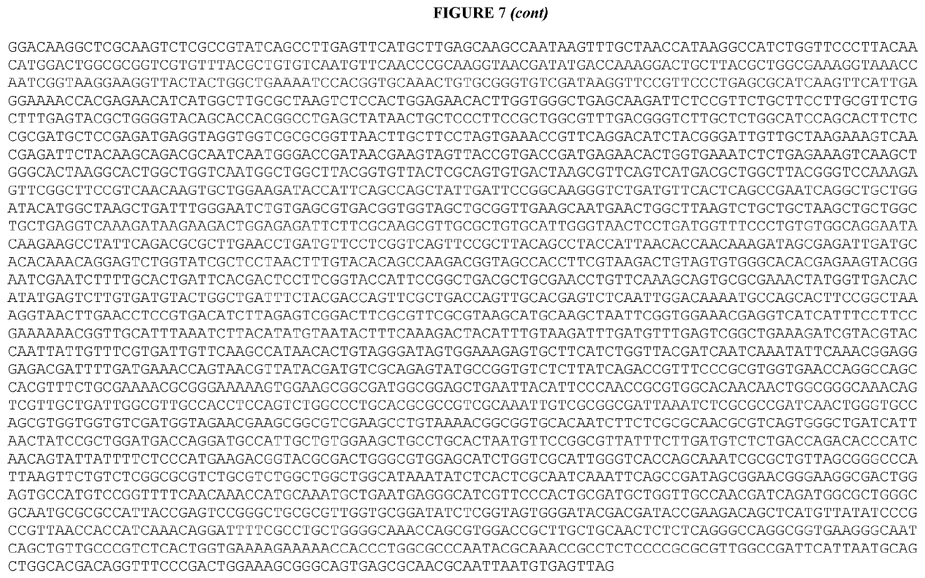

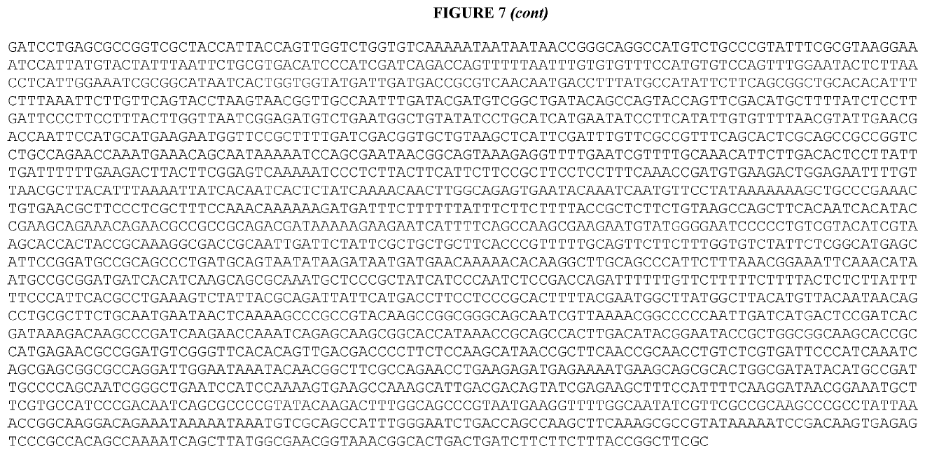

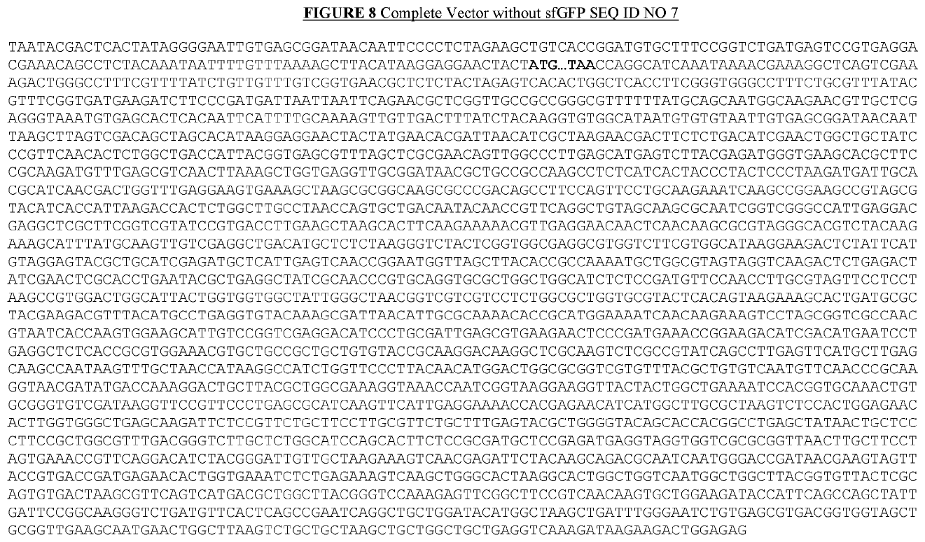

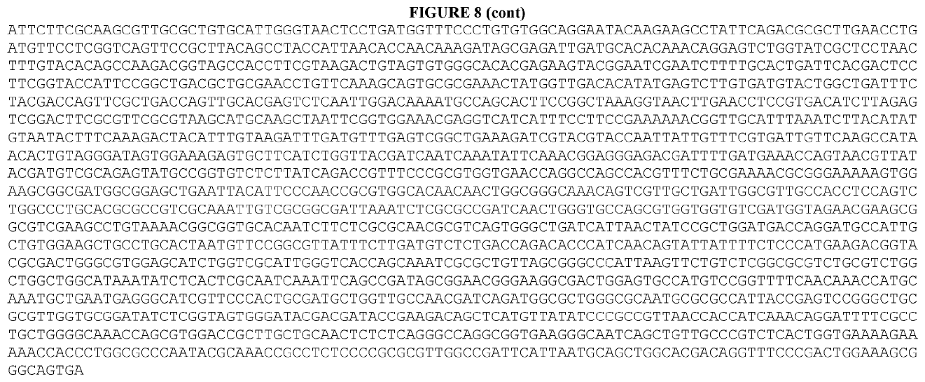

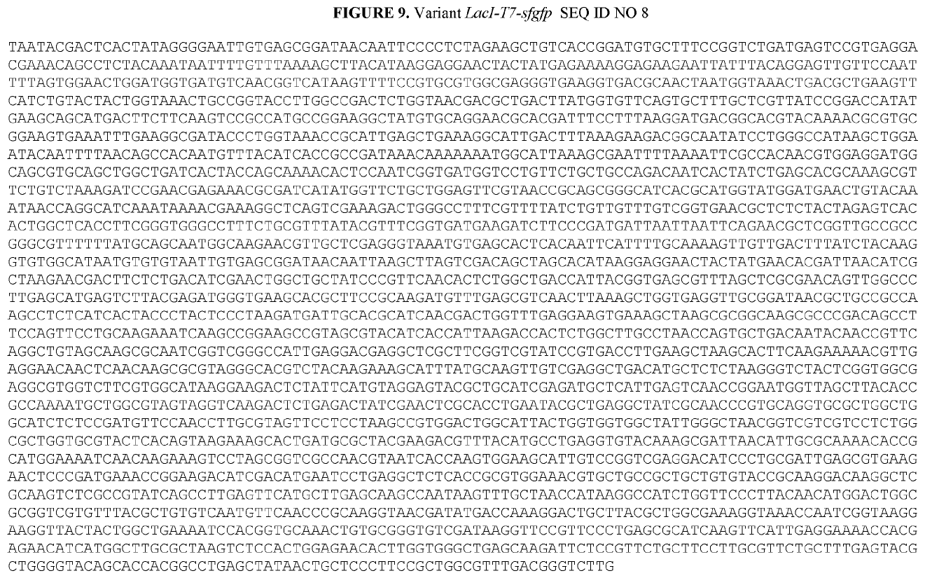

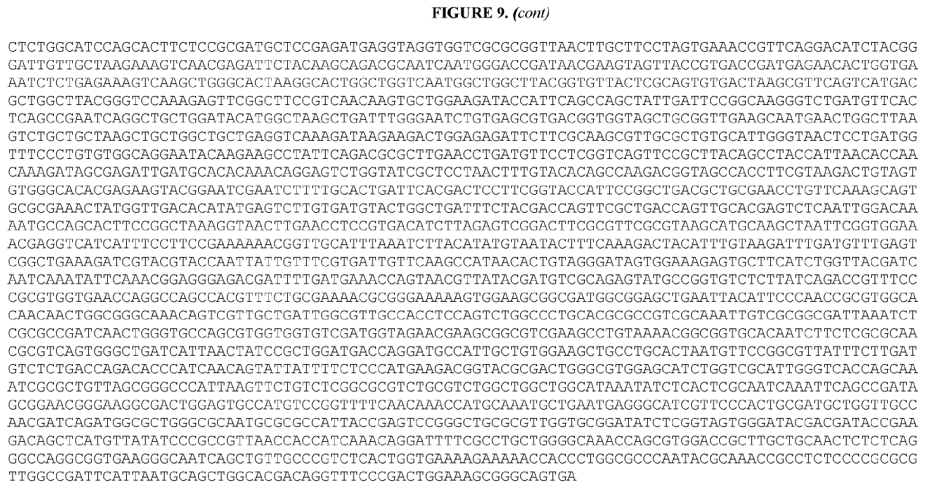

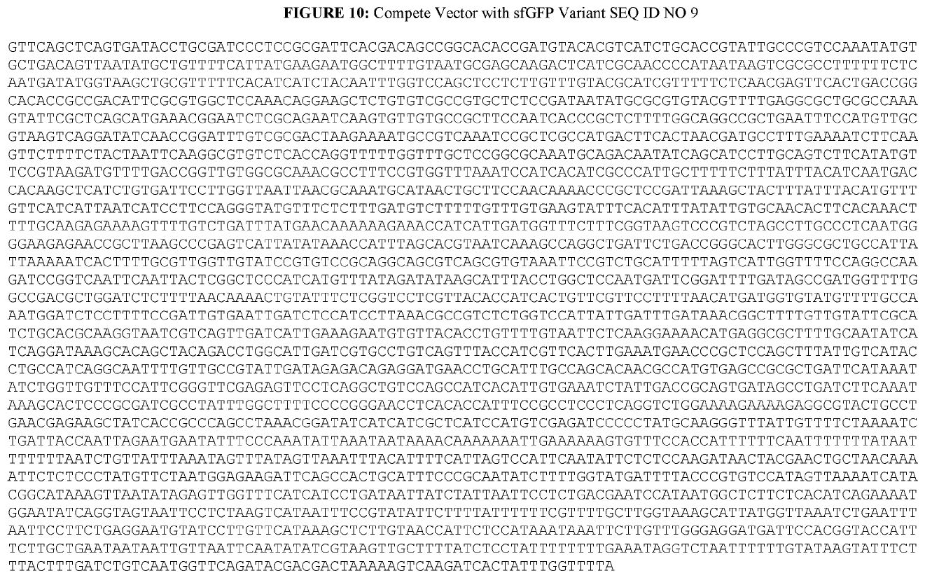

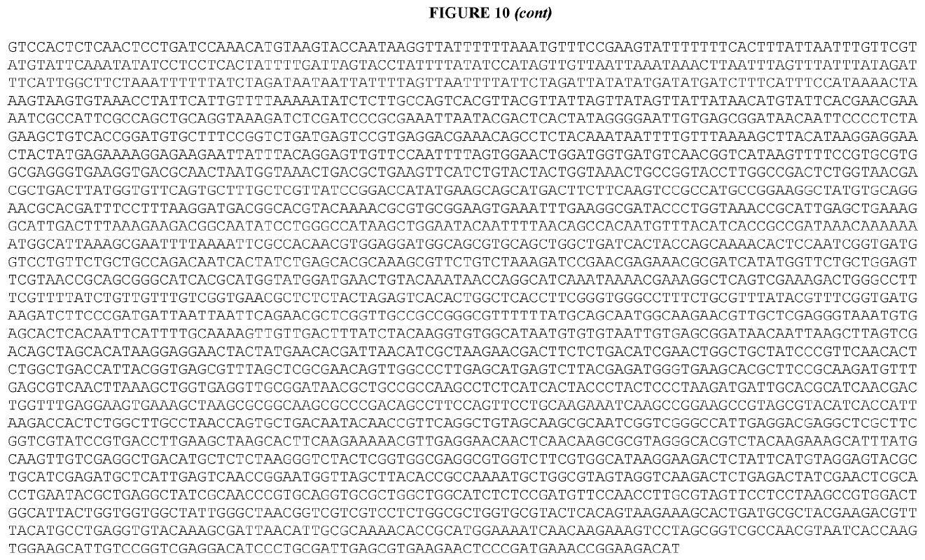

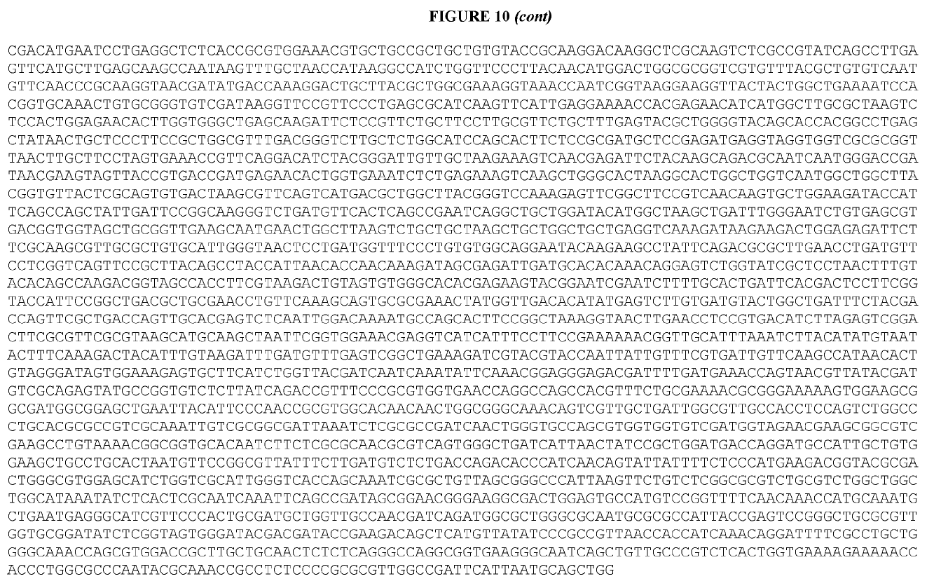

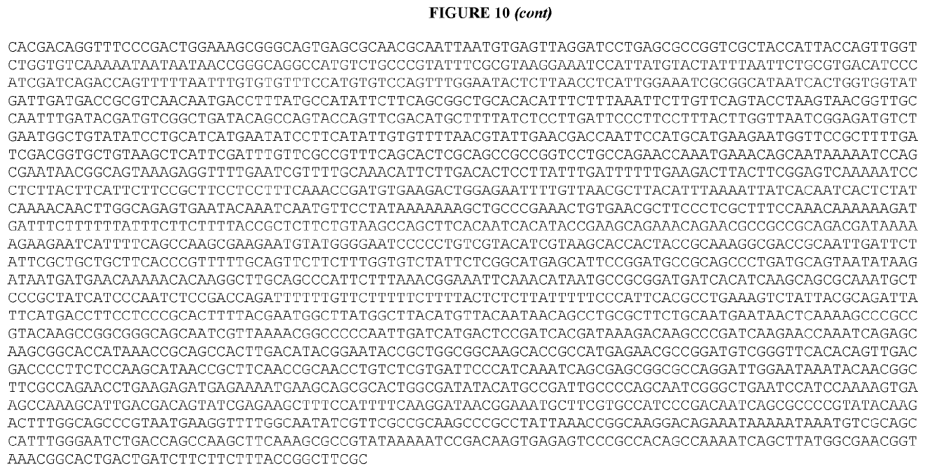

In more detail, we have engineered a stringent (i.e. non-leaky) and highly-inducible LacI-T7 promoter system for B. subtilis. Our system utilizes the hybrid P_{T7lac} promoter to express a gene of interest, and the IPTG-inducible promoter P_{hy-spank} to express the T7 RNA Polymerase (T7 RNAP) (FIG. 1A).

In the absence of the IPTG inducer, the repressor lacI is active and both T7 RNAP and P_{T7lac} are thereby repressed. This dual repression minimizes leaky expression of the gene of interest. Thus, the system is very stringent, with little to no detectable leaking. Upon addition of IPTG, LacI activity is inhibited, and the newly produced T7 RNAP strongly transcribes the gene of interest from the now de-repressed P_{T7lac} (FIG. 1A).

Our LacI-T7 design is conceptually similar to some variants of the commercial E. coli pET expression system, where IPTG also induces both expression of T7 RNAP and de-repression of P_{T7lac}. However, our system uses Bacillus or B. subtilis-specific promoters (other than P_{T7lac}) and ribosome-binding sites (RBSs). Additionally, while a few B. subtilis gene expression systems based on T7 RNAP have been previously reported, they use the LacI-independent Pr7 instead of P_{T7lac}, resulting in dynamic ranges of less than 50.

To validate our design, we placed a sfgfp reporter gene²² with a codon-optimized N-terminal fragment (sfgfp*) under control of LacI-T7, the self-cleaving ribozyme RiboJ, and RBS MF001, and integrated it into the non-essential amyE locus of the B. subtilis genome as a single copy (FIG. 1A).

We utilized flow cytometry to characterize sfGFP fluorescence levels in the absence and presence of IPTG. We found that, in the absence of inducer, sfGFP fluorescence equals 17.9±4.7 molecules of equivalent fluorescein (MEFL) (FIGS. 1B and C). In contrast, sfGFP levels in the presence of IPTG are 432,000±20,000 MEFL (FIGS. 1B and C), resulting in a dynamic range of 25,300±6,900. Remarkably, only a small growth slowdown was observed under inducing conditions (cell division time: 25.15±0.46 minutes without IPTG, 30.2±1.1 minutes with IPTG) (not shown). Polyacrylamide gel electrophoresis (PAGE) analysis of total cellular protein indicated that sfGFP is the mostly highly expressed protein in the cell in the presence of IPTG (not shown).

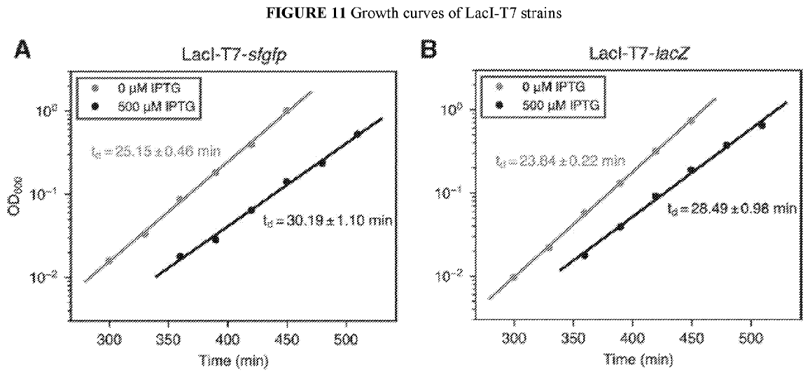

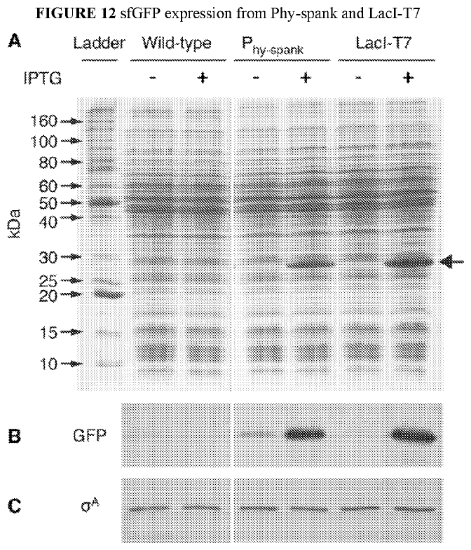

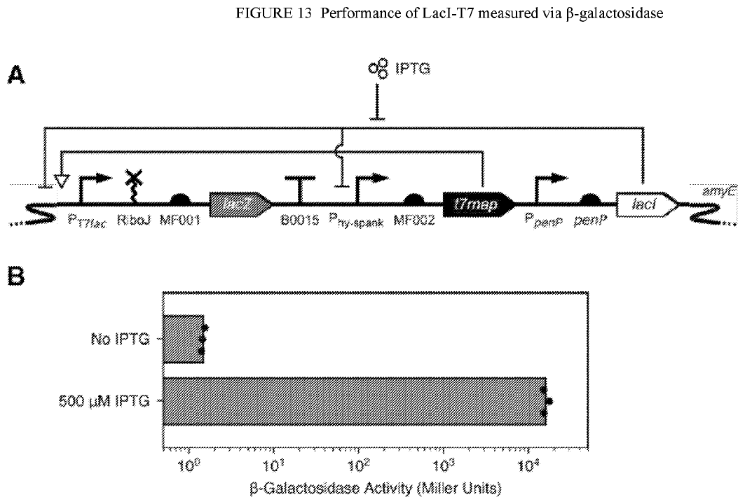

To validate that this high dynamic range is preserved when expressing a different gene of interest, we placed the lacZ reporter gene under LacI-T7 (FIG. 13) and measured β-galactosidase activity in the absence and presence of IPTG. This resulted in a dynamic range of 11,000±1,200 (FIG. 11-12). The difference between this value and the one obtained with sfGFP might be explained by uncertainty in reporter measurements under non-inducing conditions, which result in signals that are close to background. Again, we observed only a mild growth slowdown (cell division time: 23.84±0.22 minutes without IPTG, 28.49±0.98 minutes with IPTG). We conclude that LacI-T7 can regulate expression of several genes with a dynamic range greater than 10,000-fold with little toxicity.

P_{hy-spank} is a variant of the IPTG-inducible promoter P_{spac} optimized for higher expression and dynamic range, and is perhaps the most widely used B. subtilis inducible promoter system. We constructed a second B. subtilis strain wherein sfgfp* was expressed under the control of P_{hy-spank}, RiboJ, and RBS MF001 to compare its performance to that of LacI-T7. We found that P_{hy-spank} exhibits much greater leakiness (565±18 MEFL) and a lower maximal output (160,000±14,000 MEFL) than LacI-T7, resulting in a dynamic range of only 282±18 (FIG. 1B, D).

Next, we measured the steady state transfer function of both systems by growing the corresponding strains under different concentrations of IPTG. In both cases, sfGFP fluorescence increases as a function of IPTG concentration in a manner well-approximated by a Hill function (FIG. 1E). Remarkably, both systems exhibit similar IPTG detection thresholds (50% activation concentrations: LacI-T7: 54.1±1.6 μM, P_{hy-spank}: 50.2±1.5 μM). On the other hand, the Hill coefficient of the LacI-T7 system is larger (3.377±0.051, compared to 2.120±0.033 for P_{hy-spank}), indicating that protein expression is more sensitive to changes in IPTG levels in the responsive range of the system.

Finally, we characterized the response dynamics of both systems after an instantaneous addition of saturating IPTG (FIG. 1F). As expected, both responses show an exponential-like increase until saturation. However, LacI-T7 responds slightly slower (t_{1/2}=82.1±6.5 min for LacI-T7, 62.1±1.1 min for P_{hy-spank}), consistent with the need to produce an intermediate protein (T7RNAP) before expression of the reporter gene. In conclusion, LacI-T7 exhibits lower leakiness, higher maximal expression output, similar sensitivity to inducer, and much higher dynamic range than the widely used P_{hy-spank} system, albeit with a slightly slower response time.

The superior performance of LacI-T7 appears to arise from its unique design features. First, high maximal expression results from the use of T7 RNAP, a strong viral RNA polymerase which is capable of re-directing all bacterial resources towards expression of a single output gene. In contrast, previous expression systems based on endogenous promoters are limited by the native transcriptional machinery and are subject to competition with other endogenous promoters. Second, leaky expression in the absence of inducer is reduced via the dual repression activity of LacI. In E. coli, a similar design has been shown to reduce basal expression by more than an order of magnitude compared to an unmodified PT7 output promoter.

LacI-T7 should be useful in quantitative studies of B. subtilis biology. In particular, its stable single-copy chromosomal location and its low leakiness are desirable for analyses of ultrasensitive or excitable networks where low amounts of excess protein can cause cells to undergo dramatically different differentiation programs.

We also expect LacI-T7 to be useful for heterologous protein expression applications. High expression from P_{T7lac} should enable high yields of both cytoplasmic and secreted proteins. Additionally, low leakiness in the absence of inducer should allow for fast initial cell growth, even with potentially toxic proteins. Furthermore, LacI-T7 is integrated into the B. subtilis genome, and thus will not suffer from plasmid instability issues or require strong selective pressure to be maintained. Because it relies on the orthogonal T7 polymerase, LacI-T7 could also be ported to other industrially-relevant Bacillus species or strains with little additional work.

### DNA Assembly and Cloning

All cloning and experiments were performed in B. subtilis strain PY79. Primers were ordered from Integrated DNA Technologies, Inc. P_{hy-spank} was amplified from integration plasmid pDR111. P_{T7lac} was constructed via oligo annealing and extension. Synthetic RBS MF001 was obtained from integration plasmid pMF35. Genomic homology fragments required for chromosomal integration were amplified from the purified genome of B. subtilis PY79.

All systems were built as linear double-stranded integration module (IM) or cassette, as we have previously described. Our IMs contain the DNA of interest and a selection marker flanked by 1.5 kb-long sequences homologous to the amyE locus of the B. subtilis genome where chromosomal integration via double crossover occurs. IMs were assembled from PCR-amplified parts using Golden Gate. The resulting Golden Gate product was amplified using NEB Phusion DNA Polymerase and gel purified to obtain the IM. 500 ng was transformed into competent B. subtilis using standard transformation methods.

The transformants were plated on selective media. Colonies were picked the next day and grown in LB media at 37° C. and 250 RPM for a few hours. Finally, freezer stocks were prepared with 700 μL culture and 300 μL 60% glycerol, and stored at −80° C. This method avoids sub-cloning of integration plasmids in E. coli, as long as enough PCR-amplified DNA can be obtained. The complete sequences of all IMs constructed in this study can be found in GenBank via the following accession numbers: P_{hy-spank}-sfgfp: MN005205, LacI-T7-sfgfp: MN005204, LacI-T7-lacZ: MN005206.

For DNA sequence verification, an overnight LB culture was grown from a freezer stock, and 2 μL saturated culture was used as template for a 50 μL PCR reaction, either with Taq or Phusion DNA Polymerase. PCR products obtained in this fashion were gel-purified and sent for sequence verification to Genewiz, Inc.

### Media and Experimental Protocols

We used a modified M9 medium for all experiments. 1 L 5×M9 salts at PH˜6.8 were prepared with 64 g Na₂HPO₄·7H₂O, 15 g KH₂PO₄, 2.5 g NaCl, 5 g NH₄Cl, 9.2 mL 6M HCl, and up to 1 L dH₂O. For 1 L M9, we used 200 mL 5×M9 salts, 20 mL 10% casamino acids, 6.67 mL 60% glycerol, 1 mL 50 mM FeCl₃/100 mM C₆H₈O₇ solution, 2 mL 50 mM MnSO₄, 2 mL 1M MgSO₄, 100 μL 1M CaCl₂, and dH₂O up to 1 L.

For each experiment, an overnight LB culture was started from the freezer stock of each relevant strain. The next day, saturated cultures (OD₆₀₀˜3) were diluted 10⁵-fold in M9. Media was distributed in culture tubes (3 mL per tube), inoculated with the appropriate inducers (0 μM or 500 μM IPTG), and incubated in a shaker operating at 250 rpm and 37° C., until the OD₆₀₀ reached between 0.08 and 0.15 (around 6 hours). Culture tubes were then transferred to ice. 100 μL of each sample was transferred to a flow cytometry tube containing 1 mL PBS for measurement.

### Flow Cytometry Analysis

The sfGFP fluorescence distribution of each culture was measured using a BD FACScan flow cytometer with an excitation source of 488 nm and an emission window of 510/21 nm. 30,000 events were collected per sample. A suspension of calibration beads (Spherotech® RCP-30-5A) in PBS was measured with each experiment. After data acquisition, raw .fcs flow cytometry files were processed using FlowCal.

Cell populations were gated by forward scatter/side scatter density (not shown) retaining 50% of the total number of events. Next, fluorescence of each gated event in arbitrary units was converted into standardized MEFL values using the calibration bead data. The total cellular fluorescence of each culture sample was then obtained by calculating the median MEFL fluorescence of all gated events in that sample. Finally, the reported sfGFP fluorescence values were obtained by subtracting the total cellular fluorescence of a wild-type PY79 sample measured the same day from each sample's total cellular fluorescence. Numerical sfGFP fluorescence values of every sample and replicate can be found in Table 1.

### Codon Optimization of SFGFP

Codon optimization of the N-terminal sequence of the sfgfp ORF was performed to decrease secondary structure with the RBS and increase translation efficiency. To do so, for each of the first 15 codons of the original sfgfp sequence, a synonymous codon was chosen to reduce GC and increase AU content, with A preferred over U, with no regard for codon frequency. These changes were confirmed to increase the mRNA secondary structure free energy (and thus decrease secondary structure stability) via Nupack, by using the sequence from the transcription start site up to the 90^{th} nucleotide residue of the ORF. The complete optimized sfgfp* sequence can be found in Table 2.

Statistical methods are not reproduced herein, but can be found in Castillo (2019).

Each of the following references is incorporated by reference herein in its entirety for all purposes:
Westers, L., W et al., (2004) Bacillus subtilis as cell factory for pharmaceutical proteins: a biotechnological approach to optimize the host organism. Biochim. Biophys. Acta BBA-Mol. Cell Res., 1694, 299-310.
van Dijl, J. & Hecker, M. (2013) Bacillus subtilis: from soil bacterium to super-secreting cell factory. Microb. Cell Factories, 12, 3.
Yansura, D. G. & Henner, D. J. (1984) Use of the Escherichia coli lac repressor and operator to control gene expression in Bacillus subtilis. Proc. Natl. Acad. Sci., 81, 439-443.
Kim, L., et al., (1996) A xylose-inducible Bacillus subtilis integration vector and its application. Gene, 181, 71-76.
Conrad, B., et al., (1996) A T7 promoter-specific, inducible protein expression system for Bacillus subtilis. Mol. Gen. Genet. MGG, 250, 230-236.
Bhavsar, A. P., et al., (2001) Development and Characterization of a Xylose-Dependent System for Expression of Cloned Genes in Bacillus subtilis: Conditional Complementation of a Teichoic Acid Mutant. Appl Env. Microbiol, 67, 403-410.
Bongers, R. S., et al., (2005) Development and Characterization of a Subtilin-Regulated Expression System in Bacillus subtilis: Strict Control of Gene Expression by Addition of Subtilin. Appl Env. Microbiol, 71, 8818-8824.
Chen, P. T., et al., (2010) Construction of Chromosomally Located T7 Expression System for Production of Heterologous Secreted Proteins in Bacillus subtilis. J. Agric. Food Chem., 58, 5392-5399.
Toymentseva, A. A., et al., (2012) The LIKE system, a novel protein expression toolbox for Bacillus subtilis based on the liaI promoter. Microb. Cell Factories, 11, 143.
Dubendorf, J. W. & Studier, F. W. (1991) Controlling basal expression in an inducible T7 expression system by blocking the target T7 promoter with lac repressor. J. Mol. Biol., 219, 45-59.
Guzman, L. M., et l., (1995) Tight regulation, modulation, and high-level expression by vectors containing the arabinose PBAD promoter. J. Bacteriol., 177, 4121-4130.
Lutz, R. & Bujard, H. (1997) Independent and Tight Regulation of Transcriptional Units in Escherichia Coli Via the LacR/O, the TetR/O and AraC/11-12 Regulatory Elements. Nucleic Acids Res., 25, 1203-1210.
Chen, X., L et al., (2016) An extraordinary stringent and sensitive light-switchable gene expression system for bacterial cells. Cell Res., 10.1038/cr.2016.74.
Britton, R. A., et al., (2002) Genome-Wide Analysis of the Stationary-Phase Sigma Factor (Sigma-H) Regulon of Bacillus subtilis. J. Bacteriol., 184, 4881-4890.
Studier, F. W. & Moffatt, B. A. (1986) Use of bacteriophage T7 RNA polymerase to direct selective high-level expression of cloned genes. J. Mol. Biol., 189, 113-130.
Castillo-Hair, Sebastian M., et al. “An Engineered B. subtilis Inducible Promoter System with over 10 000-Fold Dynamic Range.” ACS Synthetic Biology, 8, no. 7 (2019) American Chemical Society: 1673-1678.

TABLE 1

| Strain | IPTG (μM) | Replicate | sfGFP Fluorescence (MEFL) |
|---|---|---|---|
| Physpank-sfgfp | 0 | 1 | 12.6247004 |
| Physpank-sfgfp | 0 | 2 | 8.483803292 |
| Physpank-sfgfp | 0 | 3 | 7.244700498 |
| Physpank-sfgfp | 500 | 1 | 2429.998994 |
| Physpank-sfgfp | 500 | 2 | 2611.038636 |
| Physpank-sfgfp | 500 | 3 | 2068.52592 |
| Lacl-T7-sfgfp | 0 | 1 | −11.70963282 |
| Lacl-T7-sfgfp | 0 | 2 | −0.006598202 |
| Lacl-T7-sfgfp | 0 | 3 | −2.802232792 |
| Lacl-T7-sfgfp | 500 | 1 | 108557.0156 |
| Lacl-T7-sfgfp | 500 | 2 | 113360.4879 |
| Lacl-T7-sfgfp | 500 | 3 | 115850.8173 |

TABLE 2
VARIOUS SEQUENCES

| Name | Type | Description | SEQUENCE |
|---|---|---|---|
| sfgfp | CDS | Superfolder | ATGCGTAAAGGCGAAGAGCTGTTCACTGGTGTCGTCCCT |
| | | green | ATTCTGGTGGAACTGGATGGTGATGTCAACGGTCATAAG |
| | | fluorescent | TTTTCCGTGCGTGGCGAGGGTGAAGGTGACGCAACTAAT |
| | | protein | GGTAAACTGACGCTGAAGTTCATCTGTACTACTGGTAAA |
| | | SEQ ID NO 10 | CTGCCGGTACCTTGGCCGACTCTGGTAACGACGCTGACT |
| | | | TATGGTGTTCAGTGCTTTGCTCGTTATCCGGACCATATG |
| | | | AAGCAGCATGACTTCTTCAAGTCCGCCATGCCGGAAGGC |
| | | | TATGTGCAGGAACGCACGATTTCCTTTAAGGATGACGGC |
| | | | ACGTACAAAACGCGTGCGGAAGTGAAATTTGAAGGCGAT |
| | | | ACCCTGGTAAACCGCATTGAGCTGAAAGGCATTGACTTT |
| | | | AAAGAAGACGGCAATATCCTGGGCCATAAGCTGGAATAC |
| | | | AATTTTAACAGCCACAATGTTTACATCACCGCCGATAAA |
| | | | CAAAAAAATGGCATTAAAGCGAATTTTAAAATTCGCCAC |
| | | | AACGTGGAGGATGGCAGCGTGCAGCTGGCTGATCACTAC |
| | | | CAGCAAAACACTCCAATCGGTGATGGTCCTGTTCTGCTG |
| | | | CCAGACAATCACTATCTGAGCACGCAAAGCGTTCTGTCT |
| | | | AAAGATCCGAACGAGAAACGCGATCATATGGTTCTGCTG |
| | | | GAGTTCGTAACCGCAGCGGGCATCACGCATGGTATGGAT |
| | | | GAACTGTACAAATGATGA |
| lacl | CDS | Lactose | ATGAAACCAGTAACGTTATACGATGTCGCAGAGTATGCC |
| | | repressor | GGTGTCTCTTATCAGACCGTTTCCCGCGTGGTGAACCAG |
| | | SEQ ID NO 11 | GCCAGCCACGTTTCTGCGAAAACGCGGGAAAAAGTGGAA |
| | | | GCGGCGATGGCGGAGCTGAATTACATTCCCAACCGCGTG |
| | | | GCACAACAACTGGCGGGCAAACAGTCGTTGCTGATTGGC |
| | | | GTTGCCACCTCCAGTCTGGCCCTGCACGCGCCGTCGCAA |
| | | | ATTGTCGCGGCGATTAAATCTCGCGCCGATCAACTGGGT |
| | | | GCCAGCGTGGTGGTGTCGATGGTAGAACGAAGCGGCGTC |
| | | | GAAGCCTGTAAAACGGCGGTGCACAATCTTCTCGCGCAA |
| | | | CGCGTCAGTGGGCTGATCATTAACTATCCGCTGGATGAC |
| | | | CAGGATGCCATTGCTGTGGAAGCTGCCTGCACTAATGTT |
| | | | CCGGCGTTATTTCTTGATGTCTCTGACCAGACACCCATC |
| | | | AACAGTATTATTTTCTCCCATGAAGACGGTACGCGACTG |
| | | | GGCGTGGAGCATCTGGTCGCATTGGGTCACCAGCAAATC |
| | | | GCGCTGTTAGCGGGCCCATTAAGTTCTGTCTCGGCGCGT |
| | | | CTGCGTCTGGCTGGCTGGCATAAATATCTCACTCGCAAT |
| | | | CAAATTCAGCCGATAGCGGAACGGGAAGGCGACTGGAGT |
| | | | GCCATGTCCGGTTTTCAACAAACCATGCAAATGCTGAAT |
| | | | GAGGGCATCGTTCCCACTGCGATGCTGGTTGCCAACGAT |
| | | | CAGATGGCGCTGGGCGCAATGCGCGCCATTACCGAGTCC |
| | | | GGGCTGCGCGTTGGTGCGGATATCTCGGTAGTGGGATAC |
| | | | GACGATACCGAAGACAGCTCATGTTATATCCCGCCGTTA |
| | | | ACCACCATCAAACAGGATTTTCGCCTGCTGGGGCAAACC |
| | | | AGCGTGGACCGCTTGCTGCAACTCTCTCAGGGCCAGGCG |
| | | | GTGAAGGGCAATCAGCTGTTGCCCGTCTCACTGGTGAAA |
| | | | AGAAAAACCACCCTGGCGCCCAATACGCAAACCGCCTCT |
| | | | CCCCGCGCGTTGGCCGATTCATTAATGCAGCTGGCACGA |
| | | | CAGGTTTCCCGACTGGAAAGCGGGCAGTGA |
| t7rnap | CDS | T7 RNA | ATGAACACGATTAACATCGCTAAGAACGACTTCTCTGAC |
| | | Polymerase | ATCGAACTGGCTGCTATCCCGTTCAACACTCTGGCTGAC |
| | | SEQ ID NO 12 | CATTACGGTGAGCGTTTAGCTCGCGAACAGTTGGCCCTT |
| | | | GAGCATGAGTCTTACGAGATGGGTGAAGCACGCTTCCGC |
| | | | AAGATGTTTGAGCGTCAACTTAAAGCTGGTGAGGTTGCG |
| | | | GATAACGCTGCCGCCAAGCCTCTCATCACTACCCTACTC |
| | | | CCTAAGATGATTGCACGCATCAACGACTGGTTTGAGGAA |
| | | | GTGAAAGCTAAGCGCGGCAAGCGCCCGACAGCCTTCCAG |
| | | | TTCCTGCAAGAAATCAAGCCGGAAGCCGTAGCGTACATC |
| | | | ACCATTAAGACCACTCTGGCTTGCCTAACCAGTGCTGAC |
| | | | AATACAACCGTTCAGGCTGTAGCAAGCGCAATCGGTCGG |
| | | | GCCATTGAGGACGAGGCTCGCTTCGGTCGTATCCGTGAC |
| | | | CTTGAAGCTAAGCACTTCAAGAAAAACGTTGAGGAACAA |
| | | | CTCAACAAGCGCGTAGGGCACGTCTACAAGAAAGCATTT |
| | | | ATGCAAGTTGTCGAGGCTGACATGCTCTCTAAGGGTCTA |
| | | | CTCGGTGGCGAGGCGTGGTCTTCGTGGCATAAGGAAGAC |
| | | | TCTATTCATGTAGGAGTACGCTGCATCGAGATGCTCATT |
| | | | GAGTCAACCGGAATGGTTAGCTTACACCGCCAAAATGCT |
| | | | GGCGTAGTAGGTCAAGACTCTGAGACTATCGAACTCGCA |
| | | | CCTGAATACGCTGAGGCTATCGCAACCCGTGCAGGTGCG |
| | | | CTGGCTGGCATCTCTCCGATGTTCCAACCTTGCGTAGTT |
| | | | CCTCCTAAGCCGTGGACTGGCATTACTGGTGGTGGCTAT |
| | | | TGGGCTAACGGTCGTCGTCCTCTGGCGCTGGTGCGTACT |
| | | | CACAGTAAGAAAGCACTGATGCGCTACGAAGACGTTTAC |
| | | | ATGCCTGAGGTGTACAAAGCGATTAACATTGCGCAAAAC |
| | | | ACCGCATGGAAAATCAACAAGAAAGTCCTAGCGGTCGCC |
| | | | AACGTAATCACCAAGTGGAAGCATTGTCCGGTCGAGGAC |
| | | | ATCCCTGCGATTGAGCGTGAAGAACTCCCGATGAAACCG |
| | | | GAAGACATCGACATGAATCCTGAGGCTCTCACCGCGTGG |
| | | | AAACGTGCTGCCGCTGCTGTGTACCGCAAGGACAAGGCT |
| | | | CGCAAGTCTCGCCGTATCAGCCTTGAGTTCATGCTTGAG |
| | | | CAAGCCAATAAGTTTGCTAACCATAAGGCCATCTGGTTC |
| | | | CCTTACAACATGGACTGGCGCGGTCGTGTTTACGCTGTG |
| | | | TCAATGTTCAACCCGCAAGGTAACGATATGACCAAAGGA |
| | | | CTGCTTACGCTGGCGAAAGGTAAACCAATCGGTAAGGAA |
| | | | GGTTACTACTGGCTGAAAATCCACGGTGCAAACTGTGCG |
| | | | GGTGTCGATAAGGTTCCGTTCCCTGAGCGCATCAAGTTC |
| | | | ATTGAGGAAAACCACGAGAACATCATGGCTTGCGCTAAG |
| | | | TCTCCACTGGAGAACACTTGGTGGGCTGAGCAAGATTCT |
| | | | CCGTTCTGCTTCCTTGCGTTCTGCTTTGAGTACGCTGGG |
| | | | GTACAGCACCACGGCCTGAGCTATAACTGCTCCCTTCCG |
| | | | CTGGCGTTTGACGGGTCTTGCTCTGGCATCCAGCACTTC |
| | | | TCCGCGATGCTCCGAGATGAGGTAGGTGGTCGCGCGGTT |
| | | | AACTTGCTTCCTAGTGAAACCGTTCAGGACATCTACGGG |
| | | | ATTGTTGCTAAGAAAGTCAACGAGATTCTACAAGCAGAC |
| | | | GCAATCAATGGGACCGATAACGAAGTAGTTACCGTGACC |
| | | | GATGAGAACACTGGTGAAATCTCTGAGAAAGTCAAGCTG |
| | | | GGCACTAAGGCACTGGCTGGTCAATGGCTGGCTTACGGT |
| | | | GTTACTCGCAGTGTGACTAAGCGTTCAGTCATGACGCTG |
| | | | GCTTACGGGTCCAAAGAGTTCGGCTTCCGTCAACAAGTG |
| | | | CTGGAAGATACCATTCAGCCAGCTATTGATTCCGGCAAG |
| | | | GGTCTGATGTTCACTCAGCCGAATCAGGCTGCTGGATAC |
| | | | ATGGCTAAGCTGATTTGGGAATCTGTGAGCGTGACGGTG |
| | | | GTAGCTGCGGTTGAAGCAATGAACTGGCTTAAGTCTGCT |
| | | | GCTAAGCTGCTGGCTGCTGAGGTCAAAGATAAGAAGACT |
| | | | GGAGAGATTCTTCGCAAGCGTTGCGCTGTGCATTGGGTA |
| | | | ACTCCTGATGGTTTCCCTGTGTGGCAGGAATACAAGAAG |
| | | | CCTATTCAGACGCGCTTGAACCTGATGTTCCTCGGTCAG |
| | | | TTCCGCTTACAGCCTACCATTAACACCAACAAAGATAGC |
| | | | GAGATTGATGCACACAAACAGGAGTCTGGTATCGCTCCT |
| | | | AACTTTGTACACAGCCAAGACGGTAGCCACCTTCGTAAG |
| | | | ACTGTAGTGTGGGCACACGAGAAGTACGGAATCGAATCT |
| | | | TTTGCACTGATTCACGACTCCTTCGGTACCATTCCGGCT |
| | | | GACGCTGCGAACCTGTTCAAAGCAGTGCGCGAAACTATG |
| | | | GTTGACACATATGAGTCTTGTGATGTACTGGCTGATTTC |
| | | | TACGACCAGTTCGCTGACCAGTTGCACGAGTCTCAATTG |
| | | | GACAAAATGCCAGCACTTCCGGCTAAAGGTAACTTGAAC |
| | | | CTCCGTGACATCTTAGAGTCGGACTTCGCGTTCGCGTAA |
| Phy- | Pro- | IPTG- | GGTAAATGTGAGCACTCACAATTCATTTTGCAAAAGTTG |
| spank | moter | inducible | TTGACTTTATCTACAAGGTGTGGCATAATGTGTGTAATT |
| | | promoter | GTGAGCGGATAACAATTAAGCTTAGTCGACA |
| | | SEQ ID NO 13 | |
| PT7lac | Pro- | Hybrid T7 | TAATACGACTCACTATAGGGGAATTGTGAGCGGATAACA |
| | moter | promoter with | ATTCCCCT |
| | | lac operators | |
| | | SEQ ID NO 14 | |
| PpenP | Pro- | Promoter | CGGTGGAAACGAGGTCATCATTTCCTTCCGAAAAAACGG |
| | moter | driving | TTGCATTTAAATCTTACATATGTAATACTTTCAAAGACT |
| | | expression of | ACATTTGTAAGATTTG |
| | | lacl | |
| | | SEQ ID NO 15 | |
| MF001 | RBS | Synthetic RBS | AAGCTTACATAAGGAGGAACTACT |
| | | in pMF35 SEQ | |
| | | ID NO 16 | |
| MF002 | RBS | Slightly | GCTAGCACATAAGGAGGAACTACT |
| | | modified | |
| | | MF001 | |
| | | SEQ ID NO 17 | |
| penP | RBS | RBS driving | TTCAAACGGAGGGAGACGATTTTG |
| | | expression of | |
| | | lacl SEQ ID | |
| | | NO 18 | |
| B0015 | Termi- | Terminator | CCAGGCATCAAATAAAACGAAAGGCTCAGTCGAAAGACT |
| | nator | SEQ ID NO 19 | GGGCCTTTCGTTTTATCTGTTGTTTGTCGGTGAACGCTC |
| | | | TCTACTAGAGTCACACTGGCTCACCTTCGGGGGGCCTT |
| | | | TCTGCGTTTATA |
| sfGFP | CDS | Codon | ATGAGAAAAGGAGAAGAATTATTTACAGGAGTTGTTCCA |
| | | optimized | ATTTTAGTGGAACTGGATGGTGATGTCAACGGTCATAAG |
| | | sfGFP | TTTTCCGTGCGTGGCGAGGGTGAAGGTGACGCAACTAAT |
| | | SEQ ID NO 20. | GGTAAACTGACGCTGAAGTTCATCTGTACTACTGGTAAA |
| | | | CTGCCGGTACCTTGGCCGACTCTGGTAACGACGCTGACT |
| | | | TATGGTGTTCAGTGCTTTGCTCGTTATCCGGACCATATG |
| | | | AAGCAGCATGACTTCTTCAAGTCCGCCATGCCGGAAGGC |
| | | | TATGTGCAGGAACGCACGATTTCCTTTAAGGATGACGGC |
| | | | ACGTACAAAACGCGTGCGGAAGTGAAATTTGAAGGCGAT |
| | | | ACCCTGGTAAACCGCATTGAGCTGAAAGGCATTGACTTT |
| | | | AAAGAAGACGGCAATATCCTGGGCCATAAGCTGGAATAC |
| | | | AATTTTAACAGCCACAATGTTTACATCACCGCCGATAAA |
| | | | CAAAAAAATGGCATTAAAGCGAATTTTAAAATTCGCCAC |
| | | | AACGTGGAGGATGGCAGCGTGCAGCTGGCTGATCACTAC |
| | | | CAGCAAAACACTCCAATCGGTGATGGTCCTGTTCTGCTG |
| | | | CCAGACAATCACTATCTGAGCACGCAAAGCGTTCTGTCT |
| | | | AAAGATCCGAACGAGAAACGCGATCATATGGTTCTGCTG |
| | | | GAGTTCGTAACCGCAGCGGGCATCACGCATGGTATGGAT |
| | | | GAACTGTACAAATAA |
| lacZ | CDS | β- | ATGACCATGATTACGGATTCACTGGCCGTCGTTTTACAA |
| | | galactosidase | CGTCGTGACTGGGAAAACCCTGGCGTTACCCAACTTAAT |
| | | enzyme | CGCCTTGCAGCACATCCCCCTTTCGCCAGCTGGCGTAAT |
| | | SEQ ID NO 21 | AGCGAAGAGGCCCGCACCGATCGCCCTTCCCAACAGTTG |
| | | | CGCAGCCTGAATGGCGAATGGCGCTTTGCCTGGTTTCCG |
| | | | GCACCAGAAGCGGTGCCGGAAAGCTGGCTGGAGTGCGAT |
| | | | CTTCCTGAGGCCGATACTGTCGTCGTCCCCTCAAACTGG |
| | | | CAGATGCACGGTTACGATGCGCCCATCTACACCAACGTG |
| | | | ACCTATCCCATTACGGTCAATCCGCCGTTTGTTCCCACG |
| | | | GAGAATCCGACGGGTTGTTACTCGCTCACATTTAATGTT |
| | | | GATGAAAGCTGGCTACAGGAAGGCCAGACGCGAATTATT |
| | | | TTTGATGGCGTTAACTCGGCGTTTCATCTGTGGTGCAAC |
| | | | GGGCGCTGGGTCGGTTACGGCCAGGACAGTCGTTTGCCG |
| | | | TCTGAATTTGACCTGAGCGCATTTTTACGCGCCGGAGAA |
| | | | AACCGCCTCGCGGTGATGGTGCTGCGCTGGAGTGACGGC |
| | | | AGTTATCTGGAAGATCAGGATATGTGGCGGATGAGCGGC |
| | | | ATTTTCCGTGACGTCTCGTTGCTGCATAAACCGACTACA |
| | | | CAAATCAGCGATTTCCATGTTGCCACTCGCTTTAATGAT |
| | | | GATTTCAGCCGCGCTGTACTGGAGGCTGAAGTTCAGATG |
| | | | TGCGGCGAGTTGCGTGACTACCTACGGGTAACAGTTTCT |
| | | | TTATGGCAGGGTGAAACGCAGGTCGCCAGCGGCACCGCG |
| | | | CCTTTCGGCGGTGAAATTATCGATGAGCGTGGTGGTTAT |
| | | | GCCGATCGCGTCACACTACGTCTGAACGTCGAAAACCCG |
| | | | AAACTGTGGAGCGCCGAAATCCCGAATCTCTATCGTGCG |
| | | | GTGGTTGAACTGCACACCGCCGACGGCACGCTGATTGAA |
| | | | GCAGAAGCCTGCGATGTCGGTTTCCGCGAGGTGCGGATT |
| | | | GAAAATGGTCTGCTGCTGCTGAACGGCAAGCCGTTGCTG |
| | | | ATTCGAGGCGTTAACCGTCACGAGCATCATCCTCTGCAT |
| | | | GGTCAGGTCATGGATGAGCAGACGATGGTGCAGGATATC |
| | | | CTGCTGATGAAGCAGAACAACTTTAACGCCGTGCGCTGT |
| | | | TCGCATTATCCGAACCATCCGCTGTGGTACACGCTGTGC |
| | | | GACCGCTACGGCCTGTATGTGGTGGATGAAGCCAATATT |
| | | | GAAACCCACGGCATGGTGCCAATGAATCGTCTGACCGAT |
| | | | GATCCGCGCTGGCTACCGGCGATGAGCGAACGCGTAACG |
| | | | CGAATGGTGCAGCGCGATCGTAATCACCCGAGTGTGATC |
| | | | ATCTGGTCGCTGGGGAATGAATCAGGCCACGGCGCTAAT |
| | | | CACGACGCGCTGTATCGCTGGATCAAATCTGTCGATCCT |
| | | | TCCCGCCCGGTGCAGTATGAAGGCGGCGGAGCCGACACC |
| | | | ACGGCCACCGATATTATTTGCCCGATGTACGCGCGCGTG |
| | | | GATGAAGACCAGCCCTTCCCGGCTGTGCCGAAATGGTCC |
| | | | ATCAAAAAATGGCTTTCGCTACCTGGAGAGACGCGCCCG |
| | | | CTGATCCTTTGCGAATACGCCCACGCGATGGGTAACAGT |
| | | | CTTGGCGGTTTCGCTAAATACTGGCAGGCGTTTCGTCAG |
| | | | TATCCCCGTTTACAGGGCGGCTTCGTCTGGGACTGGGTG |
| | | | GATCAGTCGCTGATTAAATATGATGAAAACGGCAACCCG |
| | | | TGGTCGGCTTACGGCGGTGATTTTGGCGATACGCCGAAC |
| | | | GATCGCCAGTTCTGTATGAACGGTCTGGTCTTTGCCGAC |
| | | | CGCACGCCGCATCCAGCGCTGACGGAAGCAAAACACCAG |
| | | | CAGCAGTTTTTCCAGTTCCGTTTATCCGGGCAAACCATC |
| | | | GAAGTGACCAGCGAATACCTGTTCCGTCATAGCGATAAC |
| | | | GAGCTCCTGCACTGGATGGTGGCGCTGGATGGTAAGCCG |
| | | | CTGGCAAGCGGTGAAGTGCCTCTGGATGTCGCTCCACAA |
| | | | GGTAAACAGTTGATTGAACTGCCTGAACTACCGCAGCCG |
| | | | GAGAGCGCCGGGCAACTCTGGCTCACAGTACGCGTAGTG |
| | | | CAACCGAACGCGACCGCATGGTCAGAAGCCGGGCACATC |
| | | | AGCGCCTGGCAGCAGTGGCGTCTGGCGGAAAACCTCAGT |
| | | | GTGACGCTCCCCGCCGCGTCCCACGCCATCCCGCATCTG |
| | | | ACCACCAGCGAAATGGATTTTTGCATCGAGCTGGGTAAT |
| | | | AAGCGTTGGCAATTTAACCGCCAGTCAGGCTTTCTTTCA |
| | | | CAGATGTGGATTGGCGATAAAAAACAACTGCTGACGCCG |
| | | | CTGCGCGATCAGTTCACCCGTGCACCGCTGGATAACGAC |
| | | | ATTGGCGTAAGTGAAGCGACCCGCATTGACCCTAACGCC |
| | | | TGGGTCGAACGCTGGAAGGCGGCGGGCCATTACCAGGCC |
| | | | GAAGCAGCGTTGTTGCAGTGCACGGCAGATACACTTGCT |
| | | | GATGCGGTGCTGATTACGACCGCTCACGCGTGGCAGCAT |
| | | | CAGGGGAAAACCTTATTTATCAGCCGGAAAACCTACCGG |
| | | | ATTGATGGTAGTGGTCAAATGGCGATTACCGTTGATGTT |
| | | | GAAGTGGCGAGCGATACACCGCATCCGGCGCGGATTGGC |
| | | | CTGAACTGCCAGCTGGCGCAGGTAGCAGAGCGGGTAAAC |
| | | | TGGCTCGGATTAGGGCCGCAAGAAAACTATCCCGACCGC |
| | | | CTTACTGCCGCCTGTTTTGACCGCTGGGATCTGCCATTG |
| | | | TCAGACATGTATACCCCGTACGTCTTCCCGAGCGAAAAC |
| | | | GGTCTGCGCTGCGGGACGCGCGAATTGAATTATGGCCCA |
| | | | CACCAGTGGCGCGGCGACTTCCAGTTCAACATCAGCCGC |
| | | | TACAGTCAACAGCAACTGATGGAAACCAGCCATCGCCAT |
| | | | CTGCTGCACGCGGAAGAAGGCACATGGCTGAATATCGAC |
| | | | GGTTTCCATATGGGGATTGGTGGCGACGACTCCTGGAGC |
| | | | CCGTCAGTATCGGCGGAATTCCAGCTGAGCGCCGGTCGC |
| | | | TACCATTACCAGTTGGTCTGGTGTCAAAAATAA |
| RiboJ | Non- | Self-cleaving | AGCTGTCACCGGATGTGCTTTCCGGTCTGATGAGTCCGT |
| | coding | ribozyme | GAGGACGAAACAGCCTCTACAAATAATTTTGTTTAA |
| | RNA | SEQ ID NO 22 | |

1) A Bacillus promoter system, comprising one or more DNA molecules comprising:
a) a LacI repressor constitutively expressed under a first constitutive promoter;
b) a T7 phage RNA polymerase (T7 RNAP) expressed from a second promoter that is normally repressed by LacI; and
c) a third promoter sequence regulated by a T7RNAP that is normally repressed by LacI.

2) The promoter system of claim 1, further comprising an open reading frame (“ORF”) for a gene of interest inserted after said third promoter sequence, such that said ORF is regulated by said third promoter sequence.

3) The promoter system of claim 1, having at least 5,000, 10,000, 15,000 or 20,000 fold activation of expression of said ORF when induced by Isopropyl β-D-1-thiogalactopyranoside (IPTG).

4) The promoter system of claim 1, comprising one or more of the following:
a) the LacI is from or is equivalent to the sequences in pDR111 or having at least 95% nucleotide identity to SEQ ID NO 11; or
b) the first constitutive promoter is a P_{penP} promoter or a sequence having at least 95% nucleotide identity to SEQ ID NO 15; or
c) the second promoter is a P_{hy-spank} promoter or a sequence having at least 95% nucleotide identity to SEQ ID NO 13; or
d) the P_{hy-spank} promoter is from or is equivalent to the sequences in pDR111; or
e) the hybrid P_{T7lac} promoter sequence is from or is equivalent to E. coli pET plasmids; or
f) the T7 RNAP is from or is equivalent to the E. coli BL21 (DE3); or
g) the T7RNAP is a sequence having at least 95% nucleotide identity to SEQ ID NO 12
h) the third promoter is a hybrid P_{T7lac} promoter sequence or a sequence having at least 95% nucleotide identity to SEQ ID NO 14; or
i) the first constitutive promoter for the LacI repressor is a promoter from Bacillus; or
j) the T7RNAP is expressed from a Bacillus LacI repressed promoter.

5) The promoter system of claim 1, further comprising an antibiotic resistance gene.

6) The promoter system of claim, wherein parts a-c are in a single DNA cassette and further comprising a spectinomycin resistance gene within said cassette.

7) The promoter system of claim 1, wherein parts a-c are in a single DNA cassette comprising a 5′ chromosomal sequence from a Bacillus amyE gene at a first end of said cassette and a 3′ chromosomal sequence from said Bacillus amyE gene at a second end of said cassette, thereby allowing homologous recombination into a genome of Bacillus.

8) The promoter system of claim 1, further comprising one or more B. subtilis-specific ribosome-binding sites (RBSs) placed to stimulate translation from mRNAs expressed from the first, second or third promoters.

9) The promoter system of claim 1, wherein said Bacillus is a Bacillus subtilis.

10) The promoter system of claim 9, further comprising one or more B. subtilis-specific ribosome-binding sites (RBSs) placed to stimulate translation from mRNAs expressed from P_{T7lac}, P_{hy-spank}, or P_{penP} promoters, or any combination thereof.

11) The promoter system of claim 1, said cassette further comprising a self-cleaving ribozyme placed after P_{T7lac} to improve output production.

12) The promoter system of claim 1, further comprising a multiple cloning site after said third promoter.

13) The promoter system of claim 1, wherein the promoter system is an inducible promoter system.

14) The promoter system of claim 13, wherein said promoter system is inducible by IPTG.

15) An expression system for Bacillus, said system being in a Bacillus and having:
a) at least 95% nucleotide identity to the sequence of SEQ ID NO 7, said 95% identity excluding an open reading frame (ORF) added thereto; and
b) having at least 5,000, 10,000, 15,000 or 20,000 fold more expression of said ORF with Isopropyl β-D-1-thiogalactopyranoside (IPTG) induction than without IPTG induction.

16) An inducible and integrative expression system for Bacillus subtilis, said system comprising an expression cassette with the following structure:
a) a first portion of a chromosomal sequence from B. subtilis at a 5′ end of said expression cassette;
b) a second portion of said chromosomal sequence at a 3′ end of said expression cassette;
c) a LacI repressor constitutively expressed under a B. subtilis P_{penP} promoter within said expression cassette;
d) a lacI-repressible T7 phage RNA polymerase (T7 RNAP) expressed from a B. subtilis P_{hy-spank} promoter within said expression cassette;
e) a hybrid promoter activated by T7RNAP and repressed by LacI that controls expression of an open reading frame (ORF).

17) The system of claim 16, wherein said ORF encodes an output mRNA, that may or may not encode a protein.

18) The system of claim 16, comprising an ORF for a target protein and being capable of a 10,000 fold increase in expression of said ORF when induced with Isopropyl β-D-1-thiogalactopyranoside (IPTG) as compared to without IPTG.

19) A vector comprising the system of claim 16, said vector being self-reproducible in a Bacillus, Escherichia or yeast.

20) A vector comprising the system of claim 16, said vector producing at least 10,000 fold more expression of said ORF in a Bacillus transformed with said vector and induced with IPTG induction, as compared to without IPTG.

21) A recombinant Bacillus, wherein said Bacillus is transformed with the system of claim 16.

22) A recombinant Bacillus, wherein said Bacillus is transformed with the system of claim 16, said expression cassette being integrated into a chromosome of said Bacillus.

23) A method of transforming Bacillus subtilis, comprising introducing the system of claim 16, to a population of Bacillus subtilis under conditions that allow transformation and integration of said system into a chromosome of said Bacillus subtilis, and selecting for transformed and integrated Bacillus subtilis.

24) A method of producing a target protein, comprising introducing the system of claim 16, to a population of Bacillus subtilis under conditions that allow integration of said expression cassette into a genome of said Bacillus subtilis, and selecting for integrated Bacillus subtilis, growing said integrated Bacillus subtilis in a growth medium until cells reach near saturation, adding IPTG to said growth medium in an amount sufficient to induce said lac operon, continuing culturing said integrated Bacillus subtilis until said ORF is expressed, and isolating said target protein from said growth medium or said transformed Bacillus subtilis or both.

25) A DNA molecule comprising SEQ ID NO. 7 plus an added open reading frame (ORF).


SEQUENCE LISTING (22 sequences)

SEQ ID NO: 1 - DNA, 154 bp, Escherichia coli
taatacgactcactataggggaattgtgagcggataacaattcccctctagaagctgtcaccggatgtgctttccggtctgatgagtccgtgaggacgaaacagcctctacaaataattttgtttaaaagcttacataaggaggaactactatg

SEQ ID NO: 2 - DNA, 109 bp, Escherichia coli
ggtaaatgtgagcactcacaattcattttgcaaaagttgttgactttatctacaaggtgtggcataatgtgtgtaattgtgagcggataacaattaagcttagtcgaca

SEQ ID NO: 3 - protein, 883 aa, T7 PHAGE
MNTINIAKNDFSDIELAAIPFNTLADHYGERLAREQLALEHESYEMGEARFRKMFERQLKAGEVADNAAAKPLITTLLPKMIARINDWFEEVKAKRGKRPTAFQFLQEIKPEAVAYITIKTTLACLTSADNTTVQAVASAIGRAIEDEARFGRIRDLEAKHFKKNVEEQLNKRVGHVYKKAFMQVVEADMLSKGLLGGEAWSSWHKEDSIHVGVRCIEMLIESTGMVSLHRQNAGVVGQDSETIELAPEYAEAIATRAGALAGISPMFQPCVVPPKPWTGITGGGYWANGRRPLALVRTHSKKALMRYEDVYMPEVYKAINIAQNTAWKINKKVLAVANVITKWKHCPVEDIPAIEREELPMKPEDIDMNPEALTAWKRAAAAVYRKDKARKSRRISLEFMLEQANKFANHKAIWFPYNMDWRGRVYAVSMFNPQGNDMTKGLLTLAKGKPIGKEGYYWLKIHGANCAGVDKVPFPERIKFIEENHENIMACAKSPLENTWWAEQDSPFCFLAFCFEYAGVQHHGLSYNCSLPLAFDGSCSGIQHFSAMLRDEVGGRAVNLLPSETVQDIYGIVAKKVNEILQADAINGTDNEVVTVTDENTGEISEKVKLGTKALAGQWLAYGVTRSVTKRSVMTLAYGSKEFGFRQQVLEDTIQPAIDSGKGLMFTQPNQAAGYMAKLIWESVSVTVVAAVEAMNWLKSAAKLLAAEVKDKKTGEILRKRCAVHWVTPDGFPVWQEYKKPIQTRLNLMFLGQFRLQPTINTNKDSEIDAHKQESGIAPNFVHSQDGSHLRKTVVWAHEKYGIESFALIHDSFGTIPADAANLFKAVRETMVDTYESCDVLADFYDQFADQLHESQLDKMPALPAKGNLNLRDILESDFAFA

SEQ ID NO: 4 - DNA, 94 bp, Bacillus licheniformis
cggtggaaacgaggtcatcatttccttccgaaaaaacggttgcatttaaatcttacatatgtaatactttcaaagactacatttgtaagatttg

SEQ ID NO: 5 - DNA, 5152 bp, Artificial Sequence
  LacI-T7-sfgfp
taatacgactcactataggggaattgtgagcggataacaattcccctctagaaagcttacataaggaggaactactatgcgtaaaggcgaagagctgttcactggtgtcgtccctattctggtggaactggatggtgatgtcaacggtcataagttttccgtgcgtggcgagggtgaaggtgacgcaactaatggtaaactgacgctgaagttcatctgtactactggtaaactgccggtaccttggccgactctggtaacgacgctgacttatggtgttcagtgctttgctcgttatccggaccatatgaagcagcatgacttcttcaagtccgccatgccggaaggctatgtgcaggaacgcacgatttcctttaaggatgacggcacgtacaaaacgcgtgcggaagtgaaatttgaaggcgataccctggtaaaccgcattgagctgaaaggcattgactttaaagaagacggcaatatcctgggccataagctggaatacaattttaacagccacaatgtttacatcaccgccgataaacaaaaaaatggcattaaagcgaattttaaaattcgccacaacgtggaggatggcagcgtgcagctggctgatcactaccagcaaaacactccaatcggtgatggtcctgttctgctgccagacaatcactatctgagcacgcaaagcgttctgtctaaagatccgaacgagaaacgcgatcatatggttctgctggagttcgtaaccgcagcgggcatcacgcatggtatggatgaactgtacaaatgatgataataatctagaccaggcatcaaataaaacgaaaggctcagtcgaaagactgggcctttcgttttatctgttgtttgtcggtgaacgctctctactagagtcacactggctcaccttcgggtgggcctttctgcgtttatacgtttcggtgatgaagatcttcccgatgattaattaattcagaacgctcggttgccgccgggcgttttttatgcagcaatggcaagaacgttgctcgagggtaaatgtgagcactcacaattcattttgcaaaagttgttgactttatctacaaggtgtggcataatgtgtgtaattgtgagcggataacaattaagcttagtcgacagctagcacataaggaggaactactatgaacacgattaacatcgctaagaacgacttctctgacatcgaactggctgctatcccgttcaacactctggctgaccattacggtgagcgtttagctcgcgaacagttggcccttgagcatgagtcttacgagatgggtgaagcacgcttccgcaagatgtttgagcgtcaacttaaagctggtgaggttgcggataacgctgccgccaagcctctcatcactaccctactccctaagatgattgcacgcatcaacgactggtttgaggaagtgaaagctaagcgcggcaagcgcccgacagccttccagttcctgcaagaaatcaagccggaagccgtagcgtacatcaccattaagaccactctggcttgcctaaccagtgctgacaatacaaccgttcaggctgtagcaagcgcaatcggtcgggccattgaggacgaggctcgcttcggtcgtatccgtgaccttgaagctaagcacttcaagaaaaacgttgaggaacaactcaacaagcgcgtagggcacgtctacaagaaagcatttatgcaagttgtcgaggctgacatgctctctaagggtctactcggtggcgaggcgtggtcttcgtggcataaggaagactctattcatgtaggagtacgctgcatcgagatgctcattgagtcaaccggaatggttagcttacaccgccaaaatgctggcgtagtaggtcaagactctgagactatcgaactcgcacctgaatacgctgaggctatcgcaacccgtgcaggtgcgctggctggcatctctccgatgttccaaccttgcgtagttcctcctaagccgtggactggcattactggtggtggctattgggctaacggtcgtcgtcctctggcgctggtgcgtactcacagtaagaaagcactgatgcgctacgaagacgtttacatgcctgaggtgtacaaagcgattaacattgcgcaaaacaccgcatggaaaatcaacaagaaagtcctagcggtcgccaacgtaatcaccaagtggaagcattgtccggtcgaggacatccctgcgattgagcgtgaagaactcccgatgaaaccggaagacatcgacatgaatcctgaggctctcaccgcgtggaaacgtgctgccgctgctgtgtaccgcaaggacaaggctcgcaagtctcgccgtatcagccttgagttcatgcttgagcaagccaataagtttgctaaccataaggccatctggttcccttacaacatggactggcgcggtcgtgtttacgctgtgtcaatgttcaacccgcaaggtaacgatatgaccaaaggactgcttacgctggcgaaaggtaaaccaatcggtaaggaaggttactactggctgaaaatccacggtgcaaactgtgcgggtgtcgataaggttccgttccctgagcgcatcaagttcattgaggaaaaccacgagaacatcatggcttgcgctaagtctccactggagaacacttggtgggctgagcaagattctccgttctgcttccttgcgttctgctttgagtacgctggggtacagcaccacggcctgagctataactgctcccttccgctggcgtttgacgggtcttgctctggcatccagcacttctccgcgatgctccgagatgaggtaggtggtcgcgcggttaacttgcttcctagtgaaaccgttcaggacatctacgggattgttgctaagaaagtcaacgagattctacaagcagacgcaatcaatgggaccgataacgaagtagttaccgtgaccgatgagaacactggtgaaatctctgagaaagtcaagctgggcactaaggcactggctggtcaatggctggcttacggtgttactcgcagtgtgactaagcgttcagtcatgacgctggcttacgggtccaaagagttcggcttccgtcaacaagtgctggaagataccattcagccagctattgattccggcaagggtctgatgttcactcagccgaatcaggctgctggatacatggctaagctgatttgggaatctgtgagcgtgacggtggtagctgcggttgaagcaatgaactggcttaagtctgctgctaagctgctggctgctgaggtcaaagataagaagactggagagattcttcgcaagcgttgcgctgtgcattgggtaactcctgatggtttccctgtgtggcaggaatacaagaagcctattcagacgcgcttgaacctgatgttcctcggtcagttccgcttacagcctaccattaacaccaacaaagatagcgagattgatgcacacaaacaggagtctggtatcgctcctaactttgtacacagccaagacggtagccaccttcgtaagactgtagtgtgggcacacgagaagtacggaatcgaatcttttgcactgattcacgactccttcggtaccattccggctgacgctgcgaacctgttcaaagcagtgcgcgaaactatggttgacacatatgagtcttgtgatgtactggctgatttctacgaccagttcgctgaccagttgcacgagtctcaattggacaaaatgccagcacttccggctaaaggtaacttgaacctccgtgacatcttagagtcggacttcgcgttcgcgtaagcatgcaagctaattcggtggaaacgaggtcatcatttccttccgaaaaaacggttgcatttaaatcttacatatgtaatactttcaaagactacatttgtaagatttgatgtttgagtcggctgaaagatcgtacgtaccaattattgtttcgtgattgttcaagccataacactgtagggatagtggaaagagtgcttcatctggttacgatcaatcaaatattcaaacggagggagacgattttgatgaaaccagtaacgttatacgatgtcgcagagtatgccggtgtctcttatcagaccgtttcccgcgtggtgaaccaggccagccacgtttctgcgaaaacgcgggaaaaagtggaagcggcgatggcggagctgaattacattcccaaccgcgtggcacaacaactggcgggcaaacagtcgttgctgattggcgttgccacctccagtctggccctgcacgcgccgtcgcaaattgtcgcggcgattaaatctcgcgccgatcaactgggtgccagcgtggtggtgtcgatggtagaacgaagcggcgtcgaagcctgtaaaacggcggtgcacaatcttctcgcgcaacgcgtcagtgggctgatcattaactatccgctggatgaccaggatgccattgctgtggaagctgcctgcactaatgttccggcgttatttcttgatgtctctgaccagacacccatcaacagtattattttctcccatgaagacggtacgcgactgggcgtggagcatctggtcgcattgggtcaccagcaaatcgcgctgttagcgggcccattaagttctgtctcggcgcgtctgcgtctggctggctggcataaatatctcactcgcaatcaaattcagccgatagcggaacgggaaggcgactggagtgccatgtccggttttcaacaaaccatgcaaatgctgaatgagggcatcgttcccactgcgatgctggttgccaacgatcagatggcgctgggcgcaatgcgcgccattaccgagtccgggctgcgcgttggtgcggatatctcggtagtgggatacgacgataccgaagacagctcatgttatatcccgccgttaaccaccatcaaacaggattttcgcctgctggggcaaaccagcgtggaccgcttgctgcaactctctcagggccaggcggtgaagggcaatcagctgttgcccgtctcactggtgaaaagaaaaaccaccctggcgcccaatacgcaaaccgcctctccccgcgcgttggccgattcattaatgcagctggcacgacaggtttcccgactggaaagcgggcagtga

SEQ ID NO: 6 - DNA, 10641 bp, Artificial Sequence
  ARTIFICIAL VECTOR
gttcagctcagtgatacctgcgatccctccgcgattcacgacagccggcacaccgatgtacacgtcatctgcaccgtattgcccgtccaaatatgtgctgacagttaatatgctgttttcattatgaagaatggcttttgtaatgcgagcaagactcatcgcaaccccataataagtcgcgccttttttctcaatgatatggtaagctgcgtttttcacatcatctacaatttggtccagctcctcttgtttgtacgcatcgtttttctcaacgagttcactgaccggcacaccgccgacattcgcgtggctccaaacaggaagctctgtgtcgccgtgctctccgataatatgcgcgtgtacgttttgaggcgctgcgccaaagtattcgctcagcatgaaacggaatctcgcagaatcaagtgttgtgccgcttccaatcacccgctcttttggcaggccgctgaatttccatgttgcgtaagtcaggatatcaaccggatttgtcgcgactaagaaaatgccgtcaaatccgctcgccatgacttcactaacgatgcctttgaaaatcttcaagttcttttctactaattcaaggcgtgtctcaccaggtttttggtttgctccggcgcaaatgcagacaatatcagcatccttgcagtcttcatatgttccgtaagatgttttgaccggttgtggcgcaaacgcctttccgtggtttaaatccatcacatcgcccattgctttttctttatttacatcaatgaccacaagctcatctgtgattccttggttaattaacgcaaatgcataactgcttccaacaaaacccgctccgattaaagctactttatttacatgtttgttcatcattaatcatccttccagggtatgtttctctttgatgtctttttgtttgtgaagtatttcacatttatattgtgcaacacttcacaaacttttgcaagagaaaagttttgtctgatttatgaacaaaaaagaaaccatcattgatggtttctttcggtaagtcccgtctagccttgccctcaatggggaagagaaccgcttaagcccgagtcattatataaaccatttagcacgtaatcaaagccaggctgattctgaccgggcacttgggcgctgccattattaaaaatcacttttgcgttggttgtatccgtgtccgcaggcagcgtcagcgtgtaaattccgtctgcatttttagtcattggttttccaggccaagatccggtcaattcaattactcggctcccatcatgtttatagatataagcatttacctggctccaatgattcggattttgatagccgatggttttggccgacgctggatctcttttaacaaaactgtatttctcggtcctcgttacaccatcactgttcgttccttttaacatgatggtgtatgttttgccaaatggatctccttttccgattgtgaattgatctccatccttaaacgccgtctctggtccattattgatttgataaacggcttttgttgtattcgcatctgcacgcaaggtaatcgtcagttgatcattgaaagaatgtgttacacctgttttgtaattctcaaggaaaacatgaggcgcttttgcaatatcatcaggataaagcacagctacagacctggcattgatcgtgcctgtcagtttaccatcgttcacttgaaatgaacccgctccagctttattgtcatacctgccatcaggcaattttgttgccgtattgatagagacagaggatgaacctgcatttgccagcacaacgccatgtgagccgcgctgattcataaatatctggttgtttccattcgggttcgagagttcctcaggctgtccagccatcacattgtgaaatctattgaccgcagtgatagcctgatcttcaaataaagcactcccgcgatcgcctatttggcttttccccgggaacctcacaccatttccgcctccctcaggtctggaaaagaaaagaggcgtactgcctgaacgagaagctatcaccgcccagcctaaacggatatcatcatcgctcatccatgtcgagatccccctatgcaagggtttattgttttctaaaatctgattaccaattagaatgaatatttcccaaatattaaataataaaacaaaaaaattgaaaaaagtgtttccaccattttttcaatttttttataatttttttaatctgttatttaaatagtttatagttaaatttacattttcattagtccattcaatattctctccaagataactacgaactgctaacaaaattctctccctatgttctaatggagaagattcagccactgcatttcccgcaatatcttttggtatgattttacccgtgtccatagttaaaatcatacggcataaagttaatatagagttggtttcatcatcctgataattatctattaattcctctgacgaatccataatggctcttctcacatcagaaaatggaatatcaggtagtaattcctctaagtcataatttccgtatattcttttattttttcgttttgcttggtaaagcattatggttaaatctgaatttaattccttctgaggaatgtatccttgttcataaagctcttgtaaccattctccataaataaattcttgtttgggaggatgattccacggtaccatttcttgctgaataataattgttaattcaatatatcgtaagttgcttttatctcctattttttttgaaataggtctaattttttgtataagtatttctttactttgatctgtcaatggttcagatacgacgactaaaaagtcaagatcactatttggttttagtccactctcaactcctgatccaaacatgtaagtaccaataaggttattttttaaatgtttccgaagtatttttttcactttattaatttgttcgtatgtattcaaatatatcctcctcactattttgattagtacctattttatatccatagttgttaattaaataaacttaatttagtttatttatagatttcattggcttctaaattttttatctagataataattattttagttaattttattctagattatatatgatatgatctttcatttccataaaactaaagtaagtgtaaacctattcattgttttaaaaatatctcttgccagtcacgttacgttattagttatagttattataacatgtattcacgaacgaaaatcgccattcgccagctgcaggtaaagatctcgatcccgcgaaattaatacgactcactataggggaattgtgagcggataacaattcccctctagaaagcttacataaggaggaactactatgcgtaaaggcgaagagctgttcactggtgtcgtccctattctggtggaactggatggtgatgtcaacggtcataagttttccgtgcgtggcgagggtgaaggtgacgcaactaatggtaaactgacgctgaagttcatctgtactactggtaaactgccggtaccttggccgactctggtaacgacgctgacttatggtgttcagtgctttgctcgttatccggaccatatgaagcagcatgacttcttcaagtccgccatgccggaaggctatgtgcaggaacgcacgatttcctttaaggatgacggcacgtacaaaacgcgtgcggaagtgaaatttgaaggcgataccctggtaaaccgcattgagctgaaaggcattgactttaaagaagacggcaatatcctgggccataagctggaatacaattttaacagccacaatgtttacatcaccgccgataaacaaaaaaatggcattaaagcgaattttaaaattcgccacaacgtggaggatggcagcgtgcagctggctgatcactaccagcaaaacactccaatcggtgatggtcctgttctgctgccagacaatcactatctgagcacgcaaagcgttctgtctaaagatccgaacgagaaacgcgatcatatggttctgctggagttcgtaaccgcagcgggcatcacgcatggtatggatgaactgtacaaatgatgataataatctagaccaggcatcaaataaaacgaaaggctcagtcgaaagactgggcctttcgttttatctgttgtttgtcggtgaacgctctctactagagtcacactggctcaccttcgggtgggcctttctgcgtttatacgtttcggtgatgaagatcttcccgatgattaattaattcagaacgctcggttgccgccgggcgttttttatgcagcaatggcaagaacgttgctcgagggtaaatgtgagcactcacaattcattttgcaaaagttgttgactttatctacaaggtgtggcataatgtgtgtaattgtgagcggataacaattaagcttagtcgacagctagcacataaggaggaactactatgaacacgattaacatcgctaagaacgacttctctgacatcgaactggctgctatcccgttcaacactctggctgaccattacggtgagcgtttagctcgcgaacagttggcccttgagcatgagtcttacgagatgggtgaagcacgcttccgcaagatgtttgagcgtcaacttaaagctggtgaggttgcggataacgctgccgccaagcctctcatcactaccctactccctaagatgattgcacgcatcaacgactggtttgaggaagtgaaagctaagcgcggcaagcgcccgacagccttccagttcctgcaagaaatcaagccggaagccgtagcgtacatcaccattaagaccactctggcttgcctaaccagtgctgacaatacaaccgttcaggctgtagcaagcgcaatcggtcgggccattgaggacgaggctcgcttcggtcgtatccgtgaccttgaagctaagcacttcaagaaaaacgttgaggaacaactcaacaagcgcgtagggcacgtctacaagaaagcatttatgcaagttgtcgaggctgacatgctctctaagggtctactcggtggcgaggcgtggtcttcgtggcataaggaagactctattcatgtaggagtacgctgcatcgagatgctcattgagtcaaccggaatggttagcttacaccgccaaaatgctggcgtagtaggtcaagactctgagactatcgaactcgcacctgaatacgctgaggctatcgcaacccgtgcaggtgcgctggctggcatctctccgatgttccaaccttgcgtagttcctcctaagccgtggactggcattactggtggtggctattgggctaacggtcgtcgtcctctggcgctggtgcgtactcacagtaagaaagcactgatgcgctacgaagacgtttacatgcctgaggtgtacaaagcgattaacattgcgcaaaacaccgcatggaaaatcaacaagaaagtcctagcggtcgccaacgtaatcaccaagtggaagcattgtccggtcgaggacatccctgcgattgagcgtgaagaactcccgatgaaaccggaagacatcgacatgaatcctgaggctctcaccgcgtggaaacgtgctgccgctgctgtgtaccgcaaggacaaggctcgcaagtctcgccgtatcagccttgagttcatgcttgagcaagccaataagtttgctaaccataaggccatctggttcccttacaacatggactggcgcggtcgtgtttacgctgtgtcaatgttcaacccgcaaggtaacgatatgaccaaaggactgcttacgctggcgaaaggtaaaccaatcggtaaggaaggttactactggctgaaaatccacggtgcaaactgtgcgggtgtcgataaggttccgttccctgagcgcatcaagttcattgaggaaaaccacgagaacatcatggcttgcgctaagtctccactggagaacacttggtgggctgagcaagattctccgttctgcttccttgcgttctgctttgagtacgctggggtacagcaccacggcctgagctataactgctcccttccgctggcgtttgacgggtcttgctctggcatccagcacttctccgcgatgctccgagatgaggtaggtggtcgcgcggttaacttgcttcctagtgaaaccgttcaggacatctacgggattgttgctaagaaagtcaacgagattctacaagcagacgcaatcaatgggaccgataacgaagtagttaccgtgaccgatgagaacactggtgaaatctctgagaaagtcaagctgggcactaaggcactggctggtcaatggctggcttacggtgttactcgcagtgtgactaagcgttcagtcatgacgctggcttacgggtccaaagagttcggcttccgtcaacaagtgctggaagataccattcagccagctattgattccggcaagggtctgatgttcactcagccgaatcaggctgctggatacatggctaagctgatttgggaatctgtgagcgtgacggtggtagctgcggttgaagcaatgaactggcttaagtctgctgctaagctgctggctgctgaggtcaaagataagaagactggagagattcttcgcaagcgttgcgctgtgcattgggtaactcctgatggtttccctgtgtggcaggaatacaagaagcctattcagacgcgcttgaacctgatgttcctcggtcagttccgcttacagcctaccattaacaccaacaaagatagcgagattgatgcacacaaacaggagtctggtatcgctcctaactttgtacacagccaagacggtagccaccttcgtaagactgtagtgtgggcacacgagaagtacggaatcgaatcttttgcactgattcacgactccttcggtaccattccggctgacgctgcgaacctgttcaaagcagtgcgcgaaactatggttgacacatatgagtcttgtgatgtactggctgatttctacgaccagttcgctgaccagttgcacgagtctcaattggacaaaatgccagcacttccggctaaaggtaacttgaacctccgtgacatcttagagtcggacttcgcgttcgcgtaagcatgcaagctaattcggtggaaacgaggtcatcatttccttccgaaaaaacggttgcatttaaatcttacatatgtaatactttcaaagactacatttgtaagatttgatgtttgagtcggctgaaagatcgtacgtaccaattattgtttcgtgattgttcaagccataacactgtagggatagtggaaagagtgcttcatctggttacgatcaatcaaatattcaaacggagggagacgattttgatgaaaccagtaacgttatacgatgtcgcagagtatgccggtgtctcttatcagaccgtttcccgcgtggtgaaccaggccagccacgtttctgcgaaaacgcgggaaaaagtggaagcggcgatggcggagctgaattacattcccaaccgcgtggcacaacaactggcgggcaaacagtcgttgctgattggcgttgccacctccagtctggccctgcacgcgccgtcgcaaattgtcgcggcgattaaatctcgcgccgatcaactgggtgccagcgtggtggtgtcgatggtagaacgaagcggcgtcgaagcctgtaaaacggcggtgcacaatcttctcgcgcaacgcgtcagtgggctgatcattaactatccgctggatgaccaggatgccattgctgtggaagctgcctgcactaatgttccggcgttatttcttgatgtctctgaccagacacccatcaacagtattattttctcccatgaagacggtacgcgactgggcgtggagcatctggtcgcattgggtcaccagcaaatcgcgctgttagcgggcccattaagttctgtctcggcgcgtctgcgtctggctggctggcataaatatctcactcgcaatcaaattcagccgatagcggaacgggaaggcgactggagtgccatgtccggttttcaacaaaccatgcaaatgctgaatgagggcatcgttcccactgcgatgctggttgccaacgatcagatggcgctgggcgcaatgcgcgccattaccgagtccgggctgcgcgttggtgcggatatctcggtagtgggatacgacgataccgaagacagctcatgttatatcccgccgttaaccaccatcaaacaggattttcgcctgctggggcaaaccagcgtggaccgcttgctgcaactctctcagggccaggcggtgaagggcaatcagctgttgcccgtctcactggtgaaaagaaaaaccaccctggcgcccaatacgcaaaccgcctctccccgcgcgttggccgattcattaatgcagctggcacgacaggtttcccgactggaaagcgggcagtgagcgcaacgcaattaatgtgagttaggatcctgagcgccggtcgctaccattaccagttggtctggtgtcaaaaataataataaccgggcaggccatgtctgcccgtatttcgcgtaaggaaatccattatgtactatttaattctgcgtgacatcccatcgatcagaccagtttttaatttgtgtgtttccatgtgtccagtttggaatactcttaacctcattggaaatcgcggcataatcactggtggtatgattgatgaccgcgtcaacaatgacctttatgccatattcttcagcggctgcacacatttctttaaattcttgttcagtacctaagtaacggttgccaatttgatacgatgtcggctgatacagccagtaccagttcgacatgcttttatctccttgattcccttcctttacttggttaatcggagatgtctgaatggctgtatatcctgcatcatgaatatccttcatattgtgttttaacgtattgaacgaccaattccatgcatgaagaatggttccgcttttgatcgacggtgctgtaagctcattcgatttgttcgccgtttcagcactcgcagccgccggtcctgccagaaccaaatgaaacagcaataaaaatccagcgaataacggcagtaaagaggttttgaatcgttttgcaaacattcttgacactccttatttgattttttgaagacttacttcggagtcaaaaatccctcttacttcattcttccgcttcctcctttcaaaccgatgtgaagactggagaattttgttaacgcttacatttaaaattatcacaatcactctatcaaaacaacttggcagagtgaatacaaatcaatgttcctataaaaaaagctgcccgaaactgtgaacgcttccctcgctttccaaacaaaaaagatgatttcttttttatttcttcttttaccgctcttctgtaagccagcttcacaatcacataccgaagcagaaacagaacgccgccgcagacgataaaaagaagaatcattttcagccaagcgaagaatgtatggggaatccccctgtcgtacatcgtaagcaccactaccgcaaaggcgaccgcaattgattctattcgctgctgcttcacccgtttttgcagttcttctttggtgtctattctcggcatgagcattccggatgccgcagccctgatgcagtaatataagataatgatgaacaaaaacacaaggcttgcagcccattctttaaacggaaattcaaacataatgccgcggatgatcacatcaagcagcgcaaatgctcccgctatcatcccaatctccgaccagattttttgttctttttcttttactctcttatttttcccattcacgcctgaaagtctattacgcagattattcatgaccttcctcccgcacttttacgaatggcttatggcttacatgttacaataacagcctgcgcttctgcaatgaataactcaaaagcccgccgtacaagccggcgggcagcaatcgttaaaacggcccccaattgatcatgactccgatcacgataaagacaagcccgatcaagaaccaaatcagagcaagcggcaccataaaccgcagccacttgacatacggaataccgctggcggcaagcaccgccatgagaacgccggatgtcgggttcacacagttgacgaccccttctccaagcataaccgcttcaaccgcaacctgtctcgtgattcccatcaaatcagcgagcggcgccaggattggaataaatacaacggcttcgccagaacctgaagagatgagaaaatgaagcagcgcactggcgatatacatgccgattgccccagcaatcgggctgaatccatccaaaagtgaagccaaagcattgacgacagtatcgagaagctttccattttcaaggataacggaaatgcttcgtgccatcccgacaatcagcgccccgtatacaagactttggcagcccgtaatgaaggttttggcaatatcgttcgccgcaagcccgcctattaaaccggcaaggacagaaataaaaataaatgtcgcagccatttgggaatctgaccagccaagcttcaaagcgccgtataaaaatccgacaagtgagagtcccgccacagccaaaatcagcttatggcgaacggtaaacggcactgactgatcttcttctttaccggcttcgc

SEQ ID NO: 7 - DNA, 4501 bp, Artificial Sequence
  ARTIFICIAL VECTOR WITHOUT SFGFP
taatacgactcactataggggaattgtgagcggataacaattcccctctagaagctgtcaccggatgtgctttccggtctgatgagtccgtgaggacgaaacagcctctacaaataattttgtttaaaagcttacataaggaggaactactatgtaaccaggcatcaaataaaacgaaaggctcagtcgaaagactgggcctttcgttttatctgttgtttgtcggtgaacgctctctactagagtcacactggctcaccttcgggtgggcctttctgcgtttatacgtttcggtgatgaagatcttcccgatgattaattaattcagaacgctcggttgccgccgggcgttttttatgcagcaatggcaagaacgttgctcgagggtaaatgtgagcactcacaattcattttgcaaaagttgttgactttatctacaaggtgtggcataatgtgtgtaattgtgagcggataacaattaagcttagtcgacagctagcacataaggaggaactactatgaacacgattaacatcgctaagaacgacttctctgacatcgaactggctgctatcccgttcaacactctggctgaccattacggtgagcgtttagctcgcgaacagttggcccttgagcatgagtcttacgagatgggtgaagcacgcttccgcaagatgtttgagcgtcaacttaaagctggtgaggttgcggataacgctgccgccaagcctctcatcactaccctactccctaagatgattgcacgcatcaacgactggtttgaggaagtgaaagctaagcgcggcaagcgcccgacagccttccagttcctgcaagaaatcaagccggaagccgtagcgtacatcaccattaagaccactctggcttgcctaaccagtgctgacaatacaaccgttcaggctgtagcaagcgcaatcggtcgggccattgaggacgaggctcgcttcggtcgtatccgtgaccttgaagctaagcacttcaagaaaaacgttgaggaacaactcaacaagcgcgtagggcacgtctacaagaaagcatttatgcaagttgtcgaggctgacatgctctctaagggtctactcggtggcgaggcgtggtcttcgtggcataaggaagactctattcatgtaggagtacgctgcatcgagatgctcattgagtcaaccggaatggttagcttacaccgccaaaatgctggcgtagtaggtcaagactctgagactatcgaactcgcacctgaatacgctgaggctatcgcaacccgtgcaggtgcgctggctggcatctctccgatgttccaaccttgcgtagttcctcctaagccgtggactggcattactggtggtggctattgggctaacggtcgtcgtcctctggcgctggtgcgtactcacagtaagaaagcactgatgcgctacgaagacgtttacatgcctgaggtgtacaaagcgattaacattgcgcaaaacaccgcatggaaaatcaacaagaaagtcctagcggtcgccaacgtaatcaccaagtggaagcattgtccggtcgaggacatccctgcgattgagcgtgaagaactcccgatgaaaccggaagacatcgacatgaatcctgaggctctcaccgcgtggaaacgtgctgccgctgctgtgtaccgcaaggacaaggctcgcaagtctcgccgtatcagccttgagttcatgcttgagcaagccaataagtttgctaaccataaggccatctggttcccttacaacatggactggcgcggtcgtgtttacgctgtgtcaatgttcaacccgcaaggtaacgatatgaccaaaggactgcttacgctggcgaaaggtaaaccaatcggtaaggaaggttactactggctgaaaatccacggtgcaaactgtgcgggtgtcgataaggttccgttccctgagcgcatcaagttcattgaggaaaaccacgagaacatcatggcttgcgctaagtctccactggagaacacttggtgggctgagcaagattctccgttctgcttccttgcgttctgctttgagtacgctggggtacagcaccacggcctgagctataactgctcccttccgctggcgtttgacgggtcttgctctggcatccagcacttctccgcgatgctccgagatgaggtaggtggtcgcgcggttaacttgcttcctagtgaaaccgttcaggacatctacgggattgttgctaagaaagtcaacgagattctacaagcagacgcaatcaatgggaccgataacgaagtagttaccgtgaccgatgagaacactggtgaaatctctgagaaagtcaagctgggcactaaggcactggctggtcaatggctggcttacggtgttactcgcagtgtgactaagcgttcagtcatgacgctggcttacgggtccaaagagttcggcttccgtcaacaagtgctggaagataccattcagccagctattgattccggcaagggtctgatgttcactcagccgaatcaggctgctggatacatggctaagctgatttgggaatctgtgagcgtgacggtggtagctgcggttgaagcaatgaactggcttaagtctgctgctaagctgctggctgctgaggtcaaagataagaagactggagagattcttcgcaagcgttgcgctgtgcattgggtaactcctgatggtttccctgtgtggcaggaatacaagaagcctattcagacgcgcttgaacctgatgttcctcggtcagttccgcttacagcctaccattaacaccaacaaagatagcgagattgatgcacacaaacaggagtctggtatcgctcctaactttgtacacagccaagacggtagccaccttcgtaagactgtagtgtgggcacacgagaagtacggaatcgaatcttttgcactgattcacgactccttcggtaccattccggctgacgctgcgaacctgttcaaagcagtgcgcgaaactatggttgacacatatgagtcttgtgatgtactggctgatttctacgaccagttcgctgaccagttgcacgagtctcaattggacaaaatgccagcacttccggctaaaggtaacttgaacctccgtgacatcttagagtcggacttcgcgttcgcgtaagcatgcaagctaattcggtggaaacgaggtcatcatttccttccgaaaaaacggttgcatttaaatcttacatatgtaatactttcaaagactacatttgtaagatttgatgtttgagtcggctgaaagatcgtacgtaccaattattgtttcgtgattgttcaagccataacactgtagggatagtggaaagagtgcttcatctggttacgatcaatcaaatattcaaacggagggagacgattttgatgaaaccagtaacgttatacgatgtcgcagagtatgccggtgtctcttatcagaccgtttcccgcgtggtgaaccaggccagccacgtttctgcgaaaacgcgggaaaaagtggaagcggcgatggcggagctgaattacattcccaaccgcgtggcacaacaactggcgggcaaacagtcgttgctgattggcgttgccacctccagtctggccctgcacgcgccgtcgcaaattgtcgcggcgattaaatctcgcgccgatcaactgggtgccagcgtggtggtgtcgatggtagaacgaagcggcgtcgaagcctgtaaaacggcggtgcacaatcttctcgcgcaacgcgtcagtgggctgatcattaactatccgctggatgaccaggatgccattgctgtggaagctgcctgcactaatgttccggcgttatttcttgatgtctctgaccagacacccatcaacagtattattttctcccatgaagacggtacgcgactgggcgtggagcatctggtcgcattgggtcaccagcaaatcgcgctgttagcgggcccattaagttctgtctcggcgcgtctgcgtctggctggctggcataaatatctcactcgcaatcaaattcagccgatagcggaacgggaaggcgactggagtgccatgtccggttttcaacaaaccatgcaaatgctgaatgagggcatcgttcccactgcgatgctggttgccaacgatcagatggcgctgggcgcaatgcgcgccattaccgagtccgggctgcgcgttggtgcggatatctcggtagtgggatacgacgataccgaagacagctcatgttatatcccgccgttaaccaccatcaaacaggattttcgcctgctggggcaaaccagcgtggaccgcttgctgcaactctctcagggccaggcggtgaagggcaatcagctgttgcccgtctcactggtgaaaagaaaaaccaccctggcgcccaatacgcaaaccgcctctccccgcgcgttggccgattcattaatgcagctggcacgacaggtttcccgactggaaagcgggcagtga

SEQ ID NO: 8 - DNA, 5212 bp, Artificial Sequence
  Variant LacI-T7-sfgfp
taatacgactcactataggggaattgtgagcggataacaattcccctctagaagctgtcaccggatgtgctttccggtctgatgagtccgtgaggacgaaacagcctctacaaataattttgtttaaaagcttacataaggaggaactactatgagaaaaggagaagaattatttacaggagttgttccaattttagtggaactggatggtgatgtcaacggtcataagttttccgtgcgtggcgagggtgaaggtgacgcaactaatggtaaactgacgctgaagttcatctgtactactggtaaactgccggtaccttggccgactctggtaacgacgctgacttatggtgttcagtgctttgctcgttatccggaccatatgaagcagcatgacttcttcaagtccgccatgccggaaggctatgtgcaggaacgcacgatttcctttaaggatgacggcacgtacaaaacgcgtgcggaagtgaaatttgaaggcgataccctggtaaaccgcattgagctgaaaggcattgactttaaagaagacggcaatatcctgggccataagctggaatacaattttaacagccacaatgtttacatcaccgccgataaacaaaaaaatggcattaaagcgaattttaaaattcgccacaacgtggaggatggcagcgtgcagctggctgatcactaccagcaaaacactccaatcggtgatggtcctgttctgctgccagacaatcactatctgagcacgcaaagcgttctgtctaaagatccgaacgagaaacgcgatcatatggttctgctggagttcgtaaccgcagcgggcatcacgcatggtatggatgaactgtacaaataaccaggcatcaaataaaacgaaaggctcagtcgaaagactgggcctttcgttttatctgttgtttgtcggtgaacgctctctactagagtcacactggctcaccttcgggtgggcctttctgcgtttatacgtttcggtgatgaagatcttcccgatgattaattaattcagaacgctcggttgccgccgggcgttttttatgcagcaatggcaagaacgttgctcgagggtaaatgtgagcactcacaattcattttgcaaaagttgttgactttatctacaaggtgtggcataatgtgtgtaattgtgagcggataacaattaagcttagtcgacagctagcacataaggaggaactactatgaacacgattaacatcgctaagaacgacttctctgacatcgaactggctgctatcccgttcaacactctggctgaccattacggtgagcgtttagctcgcgaacagttggcccttgagcatgagtcttacgagatgggtgaagcacgcttccgcaagatgtttgagcgtcaacttaaagctggtgaggttgcggataacgctgccgccaagcctctcatcactaccctactccctaagatgattgcacgcatcaacgactggtttgaggaagtgaaagctaagcgcggcaagcgcccgacagccttccagttcctgcaagaaatcaagccggaagccgtagcgtacatcaccattaagaccactctggcttgcctaaccagtgctgacaatacaaccgttcaggctgtagcaagcgcaatcggtcgggccattgaggacgaggctcgcttcggtcgtatccgtgaccttgaagctaagcacttcaagaaaaacgttgaggaacaactcaacaagcgcgtagggcacgtctacaagaaagcatttatgcaagttgtcgaggctgacatgctctctaagggtctactcggtggcgaggcgtggtcttcgtggcataaggaagactctattcatgtaggagtacgctgcatcgagatgctcattgagtcaaccggaatggttagcttacaccgccaaaatgctggcgtagtaggtcaagactctgagactatcgaactcgcacctgaatacgctgaggctatcgcaacccgtgcaggtgcgctggctggcatctctccgatgttccaaccttgcgtagttcctcctaagccgtggactggcattactggtggtggctattgggctaacggtcgtcgtcctctggcgctggtgcgtactcacagtaagaaagcactgatgcgctacgaagacgtttacatgcctgaggtgtacaaagcgattaacattgcgcaaaacaccgcatggaaaatcaacaagaaagtcctagcggtcgccaacgtaatcaccaagtggaagcattgtccggtcgaggacatccctgcgattgagcgtgaagaactcccgatgaaaccggaagacatcgacatgaatcctgaggctctcaccgcgtggaaacgtgctgccgctgctgtgtaccgcaaggacaaggctcgcaagtctcgccgtatcagccttgagttcatgcttgagcaagccaataagtttgctaaccataaggccatctggttcccttacaacatggactggcgcggtcgtgtttacgctgtgtcaatgttcaacccgcaaggtaacgatatgaccaaaggactgcttacgctggcgaaaggtaaaccaatcggtaaggaaggttactactggctgaaaatccacggtgcaaactgtgcgggtgtcgataaggttccgttccctgagcgcatcaagttcattgaggaaaaccacgagaacatcatggcttgcgctaagtctccactggagaacacttggtgggctgagcaagattctccgttctgcttccttgcgttctgctttgagtacgctggggtacagcaccacggcctgagctataactgctcccttccgctggcgtttgacgggtcttgctctggcatccagcacttctccgcgatgctccgagatgaggtaggtggtcgcgcggttaacttgcttcctagtgaaaccgttcaggacatctacgggattgttgctaagaaagtcaacgagattctacaagcagacgcaatcaatgggaccgataacgaagtagttaccgtgaccgatgagaacactggtgaaatctctgagaaagtcaagctgggcactaaggcactggctggtcaatggctggcttacggtgttactcgcagtgtgactaagcgttcagtcatgacgctggcttacgggtccaaagagttcggcttccgtcaacaagtgctggaagataccattcagccagctattgattccggcaagggtctgatgttcactcagccgaatcaggctgctggatacatggctaagctgatttgggaatctgtgagcgtgacggtggtagctgcggttgaagcaatgaactggcttaagtctgctgctaagctgctggctgctgaggtcaaagataagaagactggagagattcttcgcaagcgttgcgctgtgcattgggtaactcctgatggtttccctgtgtggcaggaatacaagaagcctattcagacgcgcttgaacctgatgttcctcggtcagttccgcttacagcctaccattaacaccaacaaagatagcgagattgatgcacacaaacaggagtctggtatcgctcctaactttgtacacagccaagacggtagccaccttcgtaagactgtagtgtgggcacacgagaagtacggaatcgaatcttttgcactgattcacgactccttcggtaccattccggctgacgctgcgaacctgttcaaagcagtgcgcgaaactatggttgacacatatgagtcttgtgatgtactggctgatttctacgaccagttcgctgaccagttgcacgagtctcaattggacaaaatgccagcacttccggctaaaggtaacttgaacctccgtgacatcttagagtcggacttcgcgttcgcgtaagcatgcaagctaattcggtggaaacgaggtcatcatttccttccgaaaaaacggttgcatttaaatcttacatatgtaatactttcaaagactacatttgtaagatttgatgtttgagtcggctgaaagatcgtacgtaccaattattgtttcgtgattgttcaagccataacactgtagggatagtggaaagagtgcttcatctggttacgatcaatcaaatattcaaacggagggagacgattttgatgaaaccagtaacgttatacgatgtcgcagagtatgccggtgtctcttatcagaccgtttcccgcgtggtgaaccaggccagccacgtttctgcgaaaacgcgggaaaaagtggaagcggcgatggcggagctgaattacattcccaaccgcgtggcacaacaactggcgggcaaacagtcgttgctgattggcgttgccacctccagtctggccctgcacgcgccgtcgcaaattgtcgcggcgattaaatctcgcgccgatcaactgggtgccagcgtggtggtgtcgatggtagaacgaagcggcgtcgaagcctgtaaaacggcggtgcacaatcttctcgcgcaacgcgtcagtgggctgatcattaactatccgctggatgaccaggatgccattgctgtggaagctgcctgcactaatgttccggcgttatttcttgatgtctctgaccagacacccatcaacagtattattttctcccatgaagacggtacgcgactgggcgtggagcatctggtcgcattgggtcaccagcaaatcgcgctgttagcgggcccattaagttctgtctcggcgcgtctgcgtctggctggctggcataaatatctcactcgcaatcaaattcagccgatagcggaacgggaaggcgactggagtgccatgtccggttttcaacaaaccatgcaaatgctgaatgagggcatcgttcccactgcgatgctggttgccaacgatcagatggcgctgggcgcaatgcgcgccattaccgagtccgggctgcgcgttggtgcggatatctcggtagtgggatacgacgataccgaagacagctcatgttatatcccgccgttaaccaccatcaaacaggattttcgcctgctggggcaaaccagcgtggaccgcttgctgcaactctctcagggccaggcggtgaagggcaatcagctgttgcccgtctcactggtgaaaagaaaaaccaccctggcgcccaatacgcaaaccgcctctccccgcgcgttggccgattcattaatgcagctggcacgacaggtttcccgactggaaagcgggcagtga

SEQ ID NO: 9 - DNA, 10701 bp, Artificial Sequence
  Compete Vector with sfGFP Variant
gttcagctcagtgatacctgcgatccctccgcgattcacgacagccggcacaccgatgtacacgtcatctgcaccgtattgcccgtccaaatatgtgctgacagttaatatgctgttttcattatgaagaatggcttttgtaatgcgagcaagactcatcgcaaccccataataagtcgcgccttttttctcaatgatatggtaagctgcgtttttcacatcatctacaatttggtccagctcctcttgtttgtacgcatcgtttttctcaacgagttcactgaccggcacaccgccgacattcgcgtggctccaaacaggaagctctgtgtcgccgtgctctccgataatatgcgcgtgtacgttttgaggcgctgcgccaaagtattcgctcagcatgaaacggaatctcgcagaatcaagtgttgtgccgcttccaatcacccgctcttttggcaggccgctgaatttccatgttgcgtaagtcaggatatcaaccggatttgtcgcgactaagaaaatgccgtcaaatccgctcgccatgacttcactaacgatgcctttgaaaatcttcaagttcttttctactaattcaaggcgtgtctcaccaggtttttggtttgctccggcgcaaatgcagacaatatcagcatccttgcagtcttcatatgttccgtaagatgttttgaccggttgtggcgcaaacgcctttccgtggtttaaatccatcacatcgcccattgctttttctttatttacatcaatgaccacaagctcatctgtgattccttggttaattaacgcaaatgcataactgcttccaacaaaacccgctccgattaaagctactttatttacatgtttgttcatcattaatcatccttccagggtatgtttctctttgatgtctttttgtttgtgaagtatttcacatttatattgtgcaacacttcacaaacttttgcaagagaaaagttttgtctgatttatgaacaaaaaagaaaccatcattgatggtttctttcggtaagtcccgtctagccttgccctcaatggggaagagaaccgcttaagcccgagtcattatataaaccatttagcacgtaatcaaagccaggctgattctgaccgggcacttgggcgctgccattattaaaaatcacttttgcgttggttgtatccgtgtccgcaggcagcgtcagcgtgtaaattccgtctgcatttttagtcattggttttccaggccaagatccggtcaattcaattactcggctcccatcatgtttatagatataagcatttacctggctccaatgattcggattttgatagccgatggttttggccgacgctggatctcttttaacaaaactgtatttctcggtcctcgttacaccatcactgttcgttccttttaacatgatggtgtatgttttgccaaatggatctccttttccgattgtgaattgatctccatccttaaacgccgtctctggtccattattgatttgataaacggcttttgttgtattcgcatctgcacgcaaggtaatcgtcagttgatcattgaaagaatgtgttacacctgttttgtaattctcaaggaaaacatgaggcgcttttgcaatatcatcaggataaagcacagctacagacctggcattgatcgtgcctgtcagtttaccatcgttcacttgaaatgaacccgctccagctttattgtcatacctgccatcaggcaattttgttgccgtattgatagagacagaggatgaacctgcatttgccagcacaacgccatgtgagccgcgctgattcataaatatctggttgtttccattcgggttcgagagttcctcaggctgtccagccatcacattgtgaaatctattgaccgcagtgatagcctgatcttcaaataaagcactcccgcgatcgcctatttggcttttccccgggaacctcacaccatttccgcctccctcaggtctggaaaagaaaagaggcgtactgcctgaacgagaagctatcaccgcccagcctaaacggatatcatcatcgctcatccatgtcgagatccccctatgcaagggtttattgttttctaaaatctgattaccaattagaatgaatatttcccaaatattaaataataaaacaaaaaaattgaaaaaagtgtttccaccattttttcaatttttttataatttttttaatctgttatttaaatagtttatagttaaatttacattttcattagtccattcaatattctctccaagataactacgaactgctaacaaaattctctccctatgttctaatggagaagattcagccactgcatttcccgcaatatcttttggtatgattttacccgtgtccatagttaaaatcatacggcataaagttaatatagagttggtttcatcatcctgataattatctattaattcctctgacgaatccataatggctcttctcacatcagaaaatggaatatcaggtagtaattcctctaagtcataatttccgtatattcttttattttttcgttttgcttggtaaagcattatggttaaatctgaatttaattccttctgaggaatgtatccttgttcataaagctcttgtaaccattctccataaataaattcttgtttgggaggatgattccacggtaccatttcttgctgaataataattgttaattcaatatatcgtaagttgcttttatctcctattttttttgaaataggtctaattttttgtataagtatttctttactttgatctgtcaatggttcagatacgacgactaaaaagtcaagatcactatttggttttagtccactctcaactcctgatccaaacatgtaagtaccaataaggttattttttaaatgtttccgaagtatttttttcactttattaatttgttcgtatgtattcaaatatatcctcctcactattttgattagtacctattttatatccatagttgttaattaaataaacttaatttagtttatttatagatttcattggcttctaaattttttatctagataataattattttagttaattttattctagattatatatgatatgatctttcatttccataaaactaaagtaagtgtaaacctattcattgttttaaaaatatctcttgccagtcacgttacgttattagttatagttattataacatgtattcacgaacgaaaatcgccattcgccagctgcaggtaaagatctcgatcccgcgaaattaatacgactcactataggggaattgtgagcggataacaattcccctctagaagctgtcaccggatgtgctttccggtctgatgagtccgtgaggacgaaacagcctctacaaataattttgtttaaaagcttacataaggaggaactactatgagaaaaggagaagaattatttacaggagttgttccaattttagtggaactggatggtgatgtcaacggtcataagttttccgtgcgtggcgagggtgaaggtgacgcaactaatggtaaactgacgctgaagttcatctgtactactggtaaactgccggtaccttggccgactctggtaacgacgctgacttatggtgttcagtgctttgctcgttatccggaccatatgaagcagcatgacttcttcaagtccgccatgccggaaggctatgtgcaggaacgcacgatttcctttaaggatgacggcacgtacaaaacgcgtgcggaagtgaaatttgaaggcgataccctggtaaaccgcattgagctgaaaggcattgactttaaagaagacggcaatatcctgggccataagctggaatacaattttaacagccacaatgtttacatcaccgccgataaacaaaaaaatggcattaaagcgaattttaaaattcgccacaacgtggaggatggcagcgtgcagctggctgatcactaccagcaaaacactccaatcggtgatggtcctgttctgctgccagacaatcactatctgagcacgcaaagcgttctgtctaaagatccgaacgagaaacgcgatcatatggttctgctggagttcgtaaccgcagcgggcatcacgcatggtatggatgaactgtacaaataaccaggcatcaaataaaacgaaaggctcagtcgaaagactgggcctttcgttttatctgttgtttgtcggtgaacgctctctactagagtcacactggctcaccttcgggtgggcctttctgcgtttatacgtttcggtgatgaagatcttcccgatgattaattaattcagaacgctcggttgccgccgggcgttttttatgcagcaatggcaagaacgttgctcgagggtaaatgtgagcactcacaattcattttgcaaaagttgttgactttatctacaaggtgtggcataatgtgtgtaattgtgagcggataacaattaagcttagtcgacagctagcacataaggaggaactactatgaacacgattaacatcgctaagaacgacttctctgacatcgaactggctgctatcccgttcaacactctggctgaccattacggtgagcgtttagctcgcgaacagttggcccttgagcatgagtcttacgagatgggtgaagcacgcttccgcaagatgtttgagcgtcaacttaaagctggtgaggttgcggataacgctgccgccaagcctctcatcactaccctactccctaagatgattgcacgcatcaacgactggtttgaggaagtgaaagctaagcgcggcaagcgcccgacagccttccagttcctgcaagaaatcaagccggaagccgtagcgtacatcaccattaagaccactctggcttgcctaaccagtgctgacaatacaaccgttcaggctgtagcaagcgcaatcggtcgggccattgaggacgaggctcgcttcggtcgtatccgtgaccttgaagctaagcacttcaagaaaaacgttgaggaacaactcaacaagcgcgtagggcacgtctacaagaaagcatttatgcaagttgtcgaggctgacatgctctctaagggtctactcggtggcgaggcgtggtcttcgtggcataaggaagactctattcatgtaggagtacgctgcatcgagatgctcattgagtcaaccggaatggttagcttacaccgccaaaatgctggcgtagtaggtcaagactctgagactatcgaactcgcacctgaatacgctgaggctatcgcaacccgtgcaggtgcgctggctggcatctctccgatgttccaaccttgcgtagttcctcctaagccgtggactggcattactggtggtggctattgggctaacggtcgtcgtcctctggcgctggtgcgtactcacagtaagaaagcactgatgcgctacgaagacgtttacatgcctgaggtgtacaaagcgattaacattgcgcaaaacaccgcatggaaaatcaacaagaaagtcctagcggtcgccaacgtaatcaccaagtggaagcattgtccggtcgaggacatccctgcgattgagcgtgaagaactcccgatgaaaccggaagacatcgacatgaatcctgaggctctcaccgcgtggaaacgtgctgccgctgctgtgtaccgcaaggacaaggctcgcaagtctcgccgtatcagccttgagttcatgcttgagcaagccaataagtttgctaaccataaggccatctggttcccttacaacatggactggcgcggtcgtgtttacgctgtgtcaatgttcaacccgcaaggtaacgatatgaccaaaggactgcttacgctggcgaaaggtaaaccaatcggtaaggaaggttactactggctgaaaatccacggtgcaaactgtgcgggtgtcgataaggttccgttccctgagcgcatcaagttcattgaggaaaaccacgagaacatcatggcttgcgctaagtctccactggagaacacttggtgggctgagcaagattctccgttctgcttccttgcgttctgctttgagtacgctggggtacagcaccacggcctgagctataactgctcccttccgctggcgtttgacgggtcttgctctggcatccagcacttctccgcgatgctccgagatgaggtaggtggtcgcgcggttaacttgcttcctagtgaaaccgttcaggacatctacgggattgttgctaagaaagtcaacgagattctacaagcagacgcaatcaatgggaccgataacgaagtagttaccgtgaccgatgagaacactggtgaaatctctgagaaagtcaagctgggcactaaggcactggctggtcaatggctggcttacggtgttactcgcagtgtgactaagcgttcagtcatgacgctggcttacgggtccaaagagttcggcttccgtcaacaagtgctggaagataccattcagccagctattgattccggcaagggtctgatgttcactcagccgaatcaggctgctggatacatggctaagctgatttgggaatctgtgagcgtgacggtggtagctgcggttgaagcaatgaactggcttaagtctgctgctaagctgctggctgctgaggtcaaagataagaagactggagagattcttcgcaagcgttgcgctgtgcattgggtaactcctgatggtttccctgtgtggcaggaatacaagaagcctattcagacgcgcttgaacctgatgttcctcggtcagttccgcttacagcctaccattaacaccaacaaagatagcgagattgatgcacacaaacaggagtctggtatcgctcctaactttgtacacagccaagacggtagccaccttcgtaagactgtagtgtgggcacacgagaagtacggaatcgaatcttttgcactgattcacgactccttcggtaccattccggctgacgctgcgaacctgttcaaagcagtgcgcgaaactatggttgacacatatgagtcttgtgatgtactggctgatttctacgaccagttcgctgaccagttgcacgagtctcaattggacaaaatgccagcacttccggctaaaggtaacttgaacctccgtgacatcttagagtcggacttcgcgttcgcgtaagcatgcaagctaattcggtggaaacgaggtcatcatttccttccgaaaaaacggttgcatttaaatcttacatatgtaatactttcaaagactacatttgtaagatttgatgtttgagtcggctgaaagatcgtacgtaccaattattgtttcgtgattgttcaagccataacactgtagggatagtggaaagagtgcttcatctggttacgatcaatcaaatattcaaacggagggagacgattttgatgaaaccagtaacgttatacgatgtcgcagagtatgccggtgtctcttatcagaccgtttcccgcgtggtgaaccaggccagccacgtttctgcgaaaacgcgggaaaaagtggaagcggcgatggcggagctgaattacattcccaaccgcgtggcacaacaactggcgggcaaacagtcgttgctgattggcgttgccacctccagtctggccctgcacgcgccgtcgcaaattgtcgcggcgattaaatctcgcgccgatcaactgggtgccagcgtggtggtgtcgatggtagaacgaagcggcgtcgaagcctgtaaaacggcggtgcacaatcttctcgcgcaacgcgtcagtgggctgatcattaactatccgctggatgaccaggatgccattgctgtggaagctgcctgcactaatgttccggcgttatttcttgatgtctctgaccagacacccatcaacagtattattttctcccatgaagacggtacgcgactgggcgtggagcatctggtcgcattgggtcaccagcaaatcgcgctgttagcgggcccattaagttctgtctcggcgcgtctgcgtctggctggctggcataaatatctcactcgcaatcaaattcagccgatagcggaacgggaaggcgactggagtgccatgtccggttttcaacaaaccatgcaaatgctgaatgagggcatcgttcccactgcgatgctggttgccaacgatcagatggcgctgggcgcaatgcgcgccattaccgagtccgggctgcgcgttggtgcggatatctcggtagtgggatacgacgataccgaagacagctcatgttatatcccgccgttaaccaccatcaaacaggattttcgcctgctggggcaaaccagcgtggaccgcttgctgcaactctctcagggccaggcggtgaagggcaatcagctgttgcccgtctcactggtgaaaagaaaaaccaccctggcgcccaatacgcaaaccgcctctccccgcgcgttggccgattcattaatgcagctggcacgacaggtttcccgactggaaagcgggcagtgagcgcaacgcaattaatgtgagttaggatcctgagcgccggtcgctaccattaccagttggtctggtgtcaaaaataataataaccgggcaggccatgtctgcccgtatttcgcgtaaggaaatccattatgtactatttaattctgcgtgacatcccatcgatcagaccagtttttaatttgtgtgtttccatgtgtccagtttggaatactcttaacctcattggaaatcgcggcataatcactggtggtatgattgatgaccgcgtcaacaatgacctttatgccatattcttcagcggctgcacacatttctttaaattcttgttcagtacctaagtaacggttgccaatttgatacgatgtcggctgatacagccagtaccagttcgacatgcttttatctccttgattcccttcctttacttggttaatcggagatgtctgaatggctgtatatcctgcatcatgaatatccttcatattgtgttttaacgtattgaacgaccaattccatgcatgaagaatggttccgcttttgatcgacggtgctgtaagctcattcgatttgttcgccgtttcagcactcgcagccgccggtcctgccagaaccaaatgaaacagcaataaaaatccagcgaataacggcagtaaagaggttttgaatcgttttgcaaacattcttgacactccttatttgattttttgaagacttacttcggagtcaaaaatccctcttacttcattcttccgcttcctcctttcaaaccgatgtgaagactggagaattttgttaacgcttacatttaaaattatcacaatcactctatcaaaacaacttggcagagtgaatacaaatcaatgttcctataaaaaaagctgcccgaaactgtgaacgcttccctcgctttccaaacaaaaaagatgatttcttttttatttcttcttttaccgctcttctgtaagccagcttcacaatcacataccgaagcagaaacagaacgccgccgcagacgataaaaagaagaatcattttcagccaagcgaagaatgtatggggaatccccctgtcgtacatcgtaagcaccactaccgcaaaggcgaccgcaattgattctattcgctgctgcttcacccgtttttgcagttcttctttggtgtctattctcggcatgagcattccggatgccgcagccctgatgcagtaatataagataatgatgaacaaaaacacaaggcttgcagcccattctttaaacggaaattcaaacataatgccgcggatgatcacatcaagcagcgcaaatgctcccgctatcatcccaatctccgaccagattttttgttctttttcttttactctcttatttttcccattcacgcctgaaagtctattacgcagattattcatgaccttcctcccgcacttttacgaatggcttatggcttacatgttacaataacagcctgcgcttctgcaatgaataactcaaaagcccgccgtacaagccggcgggcagcaatcgttaaaacggcccccaattgatcatgactccgatcacgataaagacaagcccgatcaagaaccaaatcagagcaagcggcaccataaaccgcagccacttgacatacggaataccgctggcggcaagcaccgccatgagaacgccggatgtcgggttcacacagttgacgaccccttctccaagcataaccgcttcaaccgcaacctgtctcgtgattcccatcaaatcagcgagcggcgccaggattggaataaatacaacggcttcgccagaacctgaagagatgagaaaatgaagcagcgcactggcgatatacatgccgattgccccagcaatcgggctgaatccatccaaaagtgaagccaaagcattgacgacagtatcgagaagctttccattttcaaggataacggaaatgcttcgtgccatcccgacaatcagcgccccgtatacaagactttggcagcccgtaatgaaggttttggcaatatcgttcgccgcaagcccgcctattaaaccggcaaggacagaaataaaaataaatgtcgcagccatttgggaatctgaccagccaagcttcaaagcgccgtataaaaatccgacaagtgagagtcccgccacagccaaaatcagcttatggcgaacggtaaacggcactgactgatcttcttctttaccggcttcgc

SEQ ID NO: 10 - DNA, 720 bp, Artificial Sequence
  SUPER FOLDER GFP
atgcgtaaaggcgaagagctgttcactggtgtcgtccctattctggtggaactggatggtgatgtcaacggtcataagttttccgtgcgtggcgagggtgaaggtgacgcaactaatggtaaactgacgctgaagttcatctgtactactggtaaactgccggtaccttggccgactctggtaacgacgctgacttatggtgttcagtgctttgctcgttatccggaccatatgaagcagcatgacttcttcaagtccgccatgccggaaggctatgtgcaggaacgcacgatttcctttaaggatgacggcacgtacaaaacgcgtgcggaagtgaaatttgaaggcgataccctggtaaaccgcattgagctgaaaggcattgactttaaagaagacggcaatatcctgggccataagctggaatacaattttaacagccacaatgtttacatcaccgccgataaacaaaaaaatggcattaaagcgaattttaaaattcgccacaacgtggaggatggcagcgtgcagctggctgatcactaccagcaaaacactccaatcggtgatggtcctgttctgctgccagacaatcactatctgagcacgcaaagcgttctgtctaaagatccgaacgagaaacgcgatcatatggttctgctggagttcgtaaccgcagcgggcatcacgcatggtatggatgaactgtacaaatgatga

SEQ ID NO: 11 - DNA, 1083 bp, Artificial Sequence
  LACI FROM PDR111
atgaaaccagtaacgttatacgatgtcgcagagtatgccggtgtctcttatcagaccgtttcccgcgtggtgaaccaggccagccacgtttctgcgaaaacgcgggaaaaagtggaagcggcgatggcggagctgaattacattcccaaccgcgtggcacaacaactggcgggcaaacagtcgttgctgattggcgttgccacctccagtctggccctgcacgcgccgtcgcaaattgtcgcggcgattaaatctcgcgccgatcaactgggtgccagcgtggtggtgtcgatggtagaacgaagcggcgtcgaagcctgtaaaacggcggtgcacaatcttctcgcgcaacgcgtcagtgggctgatcattaactatccgctggatgaccaggatgccattgctgtggaagctgcctgcactaatgttccggcgttatttcttgatgtctctgaccagacacccatcaacagtattattttctcccatgaagacggtacgcgactgggcgtggagcatctggtcgcattgggtcaccagcaaatcgcgctgttagcgggcccattaagttctgtctcggcgcgtctgcgtctggctggctggcataaatatctcactcgcaatcaaattcagccgatagcggaacgggaaggcgactggagtgccatgtccggttttcaacaaaccatgcaaatgctgaatgagggcatcgttcccactgcgatgctggttgccaacgatcagatggcgctgggcgcaatgcgcgccattaccgagtccgggctgcgcgttggtgcggatatctcggtagtgggatacgacgataccgaagacagctcatgttatatcccgccgttaaccaccatcaaacaggattttcgcctgctggggcaaaccagcgtggaccgcttgctgcaactctctcagggccaggcggtgaagggcaatcagctgttgcccgtctcactggtgaaaagaaaaaccaccctggcgcccaatacgcaaaccgcctctccccgcgcgttggccgattcattaatgcagctggcacgacaggtttcccgactggaaagcgggcagtga

SEQ ID NO: 12 - DNA, 2652 bp, T7 PHAGE
atgaacacgattaacatcgctaagaacgacttctctgacatcgaactggctgctatcccgttcaacactctggctgaccattacggtgagcgtttagctcgcgaacagttggcccttgagcatgagtcttacgagatgggtgaagcacgcttccgcaagatgtttgagcgtcaacttaaagctggtgaggttgcggataacgctgccgccaagcctctcatcactaccctactccctaagatgattgcacgcatcaacgactggtttgaggaagtgaaagctaagcgcggcaagcgcccgacagccttccagttcctgcaagaaatcaagccggaagccgtagcgtacatcaccattaagaccactctggcttgcctaaccagtgctgacaatacaaccgttcaggctgtagcaagcgcaatcggtcgggccattgaggacgaggctcgcttcggtcgtatccgtgaccttgaagctaagcacttcaagaaaaacgttgaggaacaactcaacaagcgcgtagggcacgtctacaagaaagcatttatgcaagttgtcgaggctgacatgctctctaagggtctactcggtggcgaggcgtggtcttcgtggcataaggaagactctattcatgtaggagtacgctgcatcgagatgctcattgagtcaaccggaatggttagcttacaccgccaaaatgctggcgtagtaggtcaagactctgagactatcgaactcgcacctgaatacgctgaggctatcgcaacccgtgcaggtgcgctggctggcatctctccgatgttccaaccttgcgtagttcctcctaagccgtggactggcattactggtggtggctattgggctaacggtcgtcgtcctctggcgctggtgcgtactcacagtaagaaagcactgatgcgctacgaagacgtttacatgcctgaggtgtacaaagcgattaacattgcgcaaaacaccgcatggaaaatcaacaagaaagtcctagcggtcgccaacgtaatcaccaagtggaagcattgtccggtcgaggacatccctgcgattgagcgtgaagaactcccgatgaaaccggaagacatcgacatgaatcctgaggctctcaccgcgtggaaacgtgctgccgctgctgtgtaccgcaaggacaaggctcgcaagtctcgccgtatcagccttgagttcatgcttgagcaagccaataagtttgctaaccataaggccatctggttcccttacaacatggactggcgcggtcgtgtttacgctgtgtcaatgttcaacccgcaaggtaacgatatgaccaaaggactgcttacgctggcgaaaggtaaaccaatcggtaaggaaggttactactggctgaaaatccacggtgcaaactgtgcgggtgtcgataaggttccgttccctgagcgcatcaagttcattgaggaaaaccacgagaacatcatggcttgcgctaagtctccactggagaacacttggtgggctgagcaagattctccgttctgcttccttgcgttctgctttgagtacgctggggtacagcaccacggcctgagctataactgctcccttccgctggcgtttgacgggtcttgctctggcatccagcacttctccgcgatgctccgagatgaggtaggtggtcgcgcggttaacttgcttcctagtgaaaccgttcaggacatctacgggattgttgctaagaaagtcaacgagattctacaagcagacgcaatcaatgggaccgataacgaagtagttaccgtgaccgatgagaacactggtgaaatctctgagaaagtcaagctgggcactaaggcactggctggtcaatggctggcttacggtgttactcgcagtgtgactaagcgttcagtcatgacgctggcttacgggtccaaagagttcggcttccgtcaacaagtgctggaagataccattcagccagctattgattccggcaagggtctgatgttcactcagccgaatcaggctgctggatacatggctaagctgatttgggaatctgtgagcgtgacggtggtagctgcggttgaagcaatgaactggcttaagtctgctgctaagctgctggctgctgaggtcaaagataagaagactggagagattcttcgcaagcgttgcgctgtgcattgggtaactcctgatggtttccctgtgtggcaggaatacaagaagcctattcagacgcgcttgaacctgatgttcctcggtcagttccgcttacagcctaccattaacaccaacaaagatagcgagattgatgcacacaaacaggagtctggtatcgctcctaactttgtacacagccaagacggtagccaccttcgtaagactgtagtgtgggcacacgagaagtacggaatcgaatcttttgcactgattcacgactccttcggtaccattccggctgacgctgcgaacctgttcaaagcagtgcgcgaaactatggttgacacatatgagtcttgtgatgtactggctgatttctacgaccagttcgctgaccagttgcacgagtctcaattggacaaaatgccagcacttccggctaaaggtaacttgaacctccgtgacatcttagagtcggacttcgcgttcgcgtaa

SEQ ID NO: 13 - DNA, 109 bp, Artificial Sequence; identical to SEQ ID NO: 2
  IPTG-INDUCIBLE PROMOTER

SEQ ID NO: 14 - DNA, 47 bp, Artificial Sequence
  Hybrid T7 promoter with lac operators SEQ ID NO 14
taatacgactcactataggggaattgtgagcggataacaattcccct

SEQ ID NO: 15 - DNA, 94 bp, Bacillus licheniformis; identical to SEQ ID NO: 4

SEQ ID NO: 16 - DNA, 24 bp, Artificial Sequence
  Synthetic RBS in pMF35
aagcttacataaggaggaactact

SEQ ID NO: 17 - DNA, 24 bp, Artificial Sequence
  Slightly modified MF001
gctagcacataaggaggaactact

SEQ ID NO: 18 - DNA, 24 bp, Artificial Sequence
  RBS driving expression of lacI
ttcaaacggagggagacgattttg

SEQ ID NO: 19 - DNA, 129 bp, Artificial Sequence
  TERMINATOR
ccaggcatcaaataaaacgaaaggctcagtcgaaagactgggcctttcgttttatctgttgtttgtcggtgaacgctctctactagagtcacactggctcaccttcgggtgggcctttctgcgtttata

SEQ ID NO: 20 - DNA, 717 bp, Artificial Sequence
  Codon optimized sfGFP
atgagaaaaggagaagaattatttacaggagttgttccaattttagtggaactggatggtgatgtcaacggtcataagttttccgtgcgtggcgagggtgaaggtgacgcaactaatggtaaactgacgctgaagttcatctgtactactggtaaactgccggtaccttggccgactctggtaacgacgctgacttatggtgttcagtgctttgctcgttatccggaccatatgaagcagcatgacttcttcaagtccgccatgccggaaggctatgtgcaggaacgcacgatttcctttaaggatgacggcacgtacaaaacgcgtgcggaagtgaaatttgaaggcgataccctggtaaaccgcattgagctgaaaggcattgactttaaagaagacggcaatatcctgggccataagctggaatacaattttaacagccacaatgtttacatcaccgccgataaacaaaaaaatggcattaaagcgaattttaaaattcgccacaacgtggaggatggcagcgtgcagctggctgatcactaccagcaaaacactccaatcggtgatggtcctgttctgctgccagacaatcactatctgagcacgcaaagcgttctgtctaaagatccgaacgagaaacgcgatcatatggttctgctggagttcgtaaccgcagcgggcatcacgcatggtatggatgaactgtacaaataa

SEQ ID NO: 21 - DNA, 3075 bp, Artificial Sequence
  ]-galactosidase enzyme
atgaccatgattacggattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgctttgcctggtttccggcaccagaagcggtgccggaaagctggctggagtgcgatcttcctgaggccgatactgtcgtcgtcccctcaaactggcagatgcacggttacgatgcgcccatctacaccaacgtgacctatcccattacggtcaatccgccgtttgttcccacggagaatccgacgggttgttactcgctcacatttaatgttgatgaaagctggctacaggaaggccagacgcgaattatttttgatggcgttaactcggcgtttcatctgtggtgcaacgggcgctgggtcggttacggccaggacagtcgtttgccgtctgaatttgacctgagcgcatttttacgcgccggagaaaaccgcctcgcggtgatggtgctgcgctggagtgacggcagttatctggaagatcaggatatgtggcggatgagcggcattttccgtgacgtctcgttgctgcataaaccgactacacaaatcagcgatttccatgttgccactcgctttaatgatgatttcagccgcgctgtactggaggctgaagttcagatgtgcggcgagttgcgtgactacctacgggtaacagtttctttatggcagggtgaaacgcaggtcgccagcggcaccgcgcctttcggcggtgaaattatcgatgagcgtggtggttatgccgatcgcgtcacactacgtctgaacgtcgaaaacccgaaactgtggagcgccgaaatcccgaatctctatcgtgcggtggttgaactgcacaccgccgacggcacgctgattgaagcagaagcctgcgatgtcggtttccgcgaggtgcggattgaaaatggtctgctgctgctgaacggcaagccgttgctgattcgaggcgttaaccgtcacgagcatcatcctctgcatggtcaggtcatggatgagcagacgatggtgcaggatatcctgctgatgaagcagaacaactttaacgccgtgcgctgttcgcattatccgaaccatccgctgtggtacacgctgtgcgaccgctacggcctgtatgtggtggatgaagccaatattgaaacccacggcatggtgccaatgaatcgtctgaccgatgatccgcgctggctaccggcgatgagcgaacgcgtaacgcgaatggtgcagcgcgatcgtaatcacccgagtgtgatcatctggtcgctggggaatgaatcaggccacggcgctaatcacgacgcgctgtatcgctggatcaaatctgtcgatccttcccgcccggtgcagtatgaaggcggcggagccgacaccacggccaccgatattatttgcccgatgtacgcgcgcgtggatgaagaccagcccttcccggctgtgccgaaatggtccatcaaaaaatggctttcgctacctggagagacgcgcccgctgatcctttgcgaatacgcccacgcgatgggtaacagtcttggcggtttcgctaaatactggcaggcgtttcgtcagtatccccgtttacagggcggcttcgtctgggactgggtggatcagtcgctgattaaatatgatgaaaacggcaacccgtggtcggcttacggcggtgattttggcgatacgccgaacgatcgccagttctgtatgaacggtctggtctttgccgaccgcacgccgcatccagcgctgacggaagcaaaacaccagcagcagtttttccagttccgtttatccgggcaaaccatcgaagtgaccagcgaatacctgttccgtcatagcgataacgagctcctgcactggatggtggcgctggatggtaagccgctggcaagcggtgaagtgcctctggatgtcgctccacaaggtaaacagttgattgaactgcctgaactaccgcagccggagagcgccgggcaactctggctcacagtacgcgtagtgcaaccgaacgcgaccgcatggtcagaagccgggcacatcagcgcctggcagcagtggcgtctggcggaaaacctcagtgtgacgctccccgccgcgtcccacgccatcccgcatctgaccaccagcgaaatggatttttgcatcgagctgggtaataagcgttggcaatttaaccgccagtcaggctttctttcacagatgtggattggcgataaaaaacaactgctgacgccgctgcgcgatcagttcacccgtgcaccgctggataacgacattggcgtaagtgaagcgacccgcattgaccctaacgcctgggtcgaacgctggaaggcggcgggccattaccaggccgaagcagcgttgttgcagtgcacggcagatacacttgctgatgcggtgctgattacgaccgctcacgcgtggcagcatcaggggaaaaccttatttatcagccggaaaacctaccggattgatggtagtggtcaaatggcgattaccgttgatgttgaagtggcgagcgatacaccgcatccggcgcggattggcctgaactgccagctggcgcaggtagcagagcgggtaaactggctcggattagggccgcaagaaaactatcccgaccgccttactgccgcctgttttgaccgctgggatctgccattgtcagacatgtataccccgtacgtcttcccgagcgaaaacggtctgcgctgcgggacgcgcgaattgaattatggcccacaccagtggcgcggcgacttccagttcaacatcagccgctacagtcaacagcaactgatggaaaccagccatcgccatctgctgcacgcggaagaaggcacatggctgaatatcgacggtttccatatggggattggtggcgacgactcctggagcccgtcagtatcggcggaattccagctgagcgccggtcgctaccattaccagttggtctggtgtcaaaaataa

SEQ ID NO: 22 - DNA, 75 bp, Artificial Sequence
  Self-cleaving ribozyme
agctgtcaccggatgtgctttccggtctgatgagtccgtgaggacgaaacagcctctacaaataattttgtttaa